(19) United States
(12) Patent Application Publication
(10) Pub. No.: US 2026/0240784 A1
(43) Pub. Date: Aug. 20, 2026

(54) PHARMACEUTICAL COMPOSITIONS WITH IMPROVED DISSOLUTION

(71) Applicant: Applied Materials, Inc., Santa Clara, CA (US)

(72) Inventors: Balaji Ganapathy, Maharashtra (IN); Ankur Kadam, Maharashtra (IN); Jayesh Arun Patil, Maharashtra (IN); Vikas Gujar, Maharashtra (IN); Mousumi Das, Maharashtra (IN); Pravin K. Narwankar, Sunnyvale, CA (US); Sushilkumar Dhanaji Patil, Maharashtra (IN)

(21) Appl. No.: 19/164,188

(22) PCT Filed: Apr. 5, 2024
(86) PCT No.: PCT/US2024/023416

§ 371 Date: Sep. 10, 2025

(30) Foreign Application Priority Data
Apr. 6, 2023 (IN) 202341025926

(51) Int. Cl. A61K 9/50; A61K 31/216

(52) U.S. Cl. CPC A61K 9/501; A61K 9/5089; A61K 31/216

(57) ABSTRACT

This disclosure pertains to a coated particle that has a drug-containing core and an inorganic coating layer and methods of preparing thereof. The coated particle has an improved solubility comparing to the uncoated drug-containing core. The drug-containing core comprises an active pharmaceutical ingredient (API) with a low solubility.

### TECHNICAL FIELD

This disclosure pertains to pharmaceutical compositions with improved dissolution profiles.

### BACKGROUND

It is of great interest to the pharmaceutical industry to develop improved formulations of active pharmaceutical ingredients (APIs). Formulation can influence the stability and bioavailability of the APIs as well as other characteristics. Formulation can also influence various aspects of drug product (DP) manufacture, for example, the ease and safety of the manufacturing process.

Biopharmaceutics Classification System (BCS) II compounds have low solubility and high permeability. Hence improving their solubility or dissolution rate has the potential to improve their bioavailability. BCS IV compounds have low solubility and low permeability. In some cases, improving solubility or dissolution rate has the potential to improve their bioavailability.

### SUMMARY

This disclosure pertains to a coated particle that has a API-containing core and an inorganic coating layer that is a metal oxide (e.g., titanium oxide, aluminum oxide or zinc oxide) or metalloid oxide (e.g., silicon oxide) and methods of preparing thereof. The coated particles have an improved wettability compared to the uncoated API-containing core. In some cases, the coated particles have an improved dispersibility compared to the uncoated API-containing core. In some cases, the coated particles have an improved wettability and improved dispersibility compared to the uncoated API-containing core. The API is preferably an organic compound or biological molecule with poor solubility and/or poor wettability. In some cases, the coating increases the dissolution rate of the API.

Applicant has found that providing a suitable metal oxide or metalloid oxide coating on API particles that are poorly wettable and/or are poorly soluble and/or poorly dispersible can improve on or both of dissolution rate and wettability. Applicant has also found that while a discontinuous or very thin coating of silicon oxide can improve wettability of poorly wettable API particles, it can be difficult to deposit silicon oxide directly onto certain API particles. Thus, it can be desirable to first apply a coating (e.g., a discontinuous or very thin) of zinc oxide or titanium oxide on the particles before applying a coating (e.g., discontinuous or very thin) of silicon oxide. A continuous coating layer fully encloses the particle. A discontinuous coating layer does not fully enclose the particle. A discontinuous coating layer may have pin-holes, cracks, or gaps.

The coating layer(s) on the particles can improve one or more of dispersibility, wettability and dissolution rate. The coatings are applied by vapor phase deposition using a precursor molecule and an oxidant (e.g., ozone or water vapor). Vapor deposition of metal oxides and metalloid oxides is commonly referred to as atomic layer deposition (ALD). However, depending on a number of factors, including the surface being coated, each cycle of the deposition reaction does not necessarily deposit one atomic layer. In many cases, each the coating lawyer is discontinuous (i.e., the layer is not a pin-hole free coating layer). Thus, where the particles have, for example a coating layer of zinc oxide, the zinc oxide does not cover the entire surface of the particle. For example, it covers no more than 90%, 80%, 70%, 60%, 50%, 40%, 30% or even 20% of the surface of the particle. Thus, the coating appears patchy. In some cases, the coating layer covers nearly the entire surface of the particle but has cracks or other defects such that it is not continuous. In some embodiments, when there are two different coating layers, each layer is discontinuous. In some cases, the two different coating layers, taken together do not from continuous coating layer.

Described herein is a pharmaceutical composition, comprising: (a) a coated particle comprising an active pharmaceutical ingredient (API)-containing core and one or more discontinuous inorganic coating layers, wherein the API-containing core has a median particle size, on a volume average basis, between 0.1 μm and 20 μm, wherein each of the one or more discontinuous inorganic coating layers comprise a metal oxide or a metalloid oxide, and (b) a pharmaceutically acceptable excipient.

Also described herein is a pharmaceutical composition, comprising: (a) a coated particle comprising an fenofibrate-containing core and one or more discontinuous inorganic coating layers, wherein the fenofibrate-containing core has a median particle size, on a volume average basis, between 0.1 μm and 20 μm, wherein each of the one or more discontinuous inorganic coating layers comprise a metal oxide or metalloid oxide, and (b) a pharmaceutically acceptable excipient.

In various embodiments: the API is a BCS class II molecule or a BCS class IV molecule; the API is poorly wettable; the API is poorly soluble; the API is poorly dispersible; the coated particles are hydrophilic; the one or more inorganic coating layers comprise or consist of zinc oxide; the one or more inorganic coating layers comprise or consist of silicon oxide (e.g., preferably formed by vapor phase deposition of SiCl4 oxidized by reaction water vapor, not ozone); the one or more inorganic coating layers consist of zinc oxide or silicon oxide; the one or more inorganic coating layers comprise an inner coating layer (e.g., discontinuous coating layer) that is selected from zinc oxide and titanium oxide and an outer coating layer that is silicon oxide; the one or more discontinuous coating layers, taken together do not provide a continuous pin-hole free coating of the API-containing core; the one or more inorganic coating layers, taken together constitute 2%-7% wt/wt of the coated particles; the one or more inorganic coating layers, taken together constitute 1%-3% wt/wt of the coated particles; the core has a median particle size, on a volume average basis, between 2 μm and 20 μm and the one or more inorganic coating layers, taken together constitute 2%-7% wt/wt of the coated particles; the core has a median particle size, on a volume average basis, between 0.1 μm and 1 μm and the one or more inorganic coating layers, taken together constitute 10%-20% wt/wt of the coated particles; the coated particle has one or more or faster dissolution rate, greater wettability, and greater dispersibility compared to the uncoated API-containing core; the dissolution rate of API in the coated particle is at least 20% higher than the dissolution rate of the API in uncoated drug-containing core (e.g., wherein the solubility is measured by dissolving 120 mg of the coated or uncoated particles in water containing 0.75% Sodium lauryl sulfate (SLS) at room temperature for 30 minutes, with 75 RPM stirring); coated particle has an improved flowability comparing to uncoated drug-containing core; the uncoated particles have a water contact angle that is between 90° and 145°; uncoated core has a water contact angle that is between greater than 145°; the coated particles have a water contact angle that is less than 90°.

Also described is a method of preparing coated particles comprising active pharmaceutical ingredient (API)-containing core, a discontinuous zinc oxide coating layer and a discontinuous silicon oxide coating layer, the method comprising the sequential steps of: (a) loading particles comprising an API into the chamber of a reactor, wherein the particles have a median particle size, on a volume average basis between 0.1 μm and 1000 μm; (b1) applying vaporous or gaseous diethyl zinc to the particles in the reactor by pulsing the vaporous or gaseous diethyl zinc into the reactor; (b2) performing one or more pump-purge cycles of the reactor using an inert gas; (b3) applying vaporous or gaseous oxidant to the particles in the reactor by pulsing the oxidant into the reactor; (b4) performing one or more pump-purge cycles of the reactor using an inert gas; (c) repeating steps (b1)-(b4) at least once to create a discontinuous zinc oxide coating layer on the particles; (d1) applying vaporous or gaseous silicon tetrachloride to the particles in the reactor by pulsing the vaporous or gaseous silicon precursor into the reactor; (d2) performing one or more pump-purge cycles of the reactor using an inert gas; (d3) applying vaporous or gaseous water to the particles in the reactor by pulsing vaporous or gaseous water into the reactor; (d4) performing one or more pump-purge cycles of the reactor using an inert gas; and (e) repeating steps (d1)-(d4) at least once to create a discontinuous silicon oxide coating layer on at least a portion of the discontinuous zinc oxide coating layer.

In various embodiments of the method, the API is fenofibrate; the API is a BCS class II or BCS class IV molecule; the uncoated particles have a water contact angle that is between 90° and 145°; the uncoated particles have a water contact angle that is between greater than 145°; the coated particles have a water contact angle that is less than 90°; the zinc oxide coating is no more than 10 nm thick; the silicon oxide layer is no more than 10 nm thick; the temperature of the chamber is maintained at a temperature between 20° C. and 60° C.; the one or more discontinuous coating layers, taken together do not provide a continuous pin-hole free coating of the API-containing core; the one or more inorganic coating layers, taken together constitute 2%-7% wt/wt of the coated particles; the one or more inorganic coating layers, taken together constitute 1%-3% wt/wt of the coated particles; the core has a median particle size, on a volume average basis, between 2 μm and 20 μm and the one or more inorganic coating layers, taken together constitute 2%-7% wt/wt of the coated particles; the core has a median particle size, on a volume average basis, between 0.1 μm and 1 μm and the one or more inorganic coating layers, taken together constitute 10%-20% wt/wt of the coated particles; the particles remain in the reactor during steps (b1)-(b4), each pump-purge cycle comprises flowing the inert gas into the reactor chamber to a desired pressure and after a delay time pumping the inert gas out of the reactor until the pressure of the inert gas is below 1 torr and repeating the steps of flowing the inert gas into the reactor chamber to a desired pressure and after a delay time pumping the inert gas out of the reactor until the pressure of the inert gas is below 1 torr.

In one aspect, the disclosure is related to a pharmaceutical composition, comprising: (a) a coated particle comprising an active pharmaceutical ingredient (API)-containing core and one or more discontinuous inorganic coating layers, wherein the API-containing core has a median particle size, on a volume average basis, between 0.1 μm and 1000 μm, wherein each of the one or more discontinuous inorganic coating layers comprise a metal oxide or a metalloid oxide, and (b) a pharmaceutically acceptable excipient.

In some embodiments, the coated particle further comprises a continuous inorganic coating layer.

In some embodiments, the one or more discontinuous coating layers, taken together, do not provide a continuous pin-hole free coating of the API-containing core.

In some embodiments, the API-containing core has a median particle size, on a volume average basis, between 0.1 μm and 20 μm.

In some embodiments, the API is a BCS class II molecule or a BCS class IV molecule.

In some embodiments, the API is poorly wettable.

In some embodiments, the API is poorly dispersible.

In some embodiments, the coated particles are hydrophilic.

In some embodiments, the inorganic coating layers consist of zinc oxide and/or silicone oxide.

In some embodiments, the one or more inorganic coating layers comprise or consist of zinc oxide.

In some embodiments, the one or more inorganic coating layers comprise or consist of silicon oxide.

In some embodiments, each of the one or more inorganic coating layers consist of zinc oxide or silicon oxide.

In some embodiments, the one or more inorganic coating layers comprise a discontinuous inner coating layer that is selected from zinc oxide and titanium oxide and an outer coating layer that is silicon oxide.

In some embodiments, the one or more inorganic coating layers, taken together constitute 2%-7% wt/wt of the coated particles.

In some embodiments, the one or more inorganic coating layers, taken together constitute 1%-3% wt/wt of the coated particles.

In some embodiments, the core has a median particle size, on a volume average basis, between 2 μm and 20 μm and the one or more inorganic coating layers, taken together constitute 2%-7% wt/wt of the coated particles.

In some embodiments, the core has a median particle size, on a volume average basis, between 0.1 μm and 1 μm and the one or more inorganic coating layers, taken together constitute 10%-20% wt/wt of the coated particles.

In some embodiments, the coated particle has one or more or faster dissolution rate, greater wettability, and greater dispersibility compared to the uncoated drug-containing core.

In some embodiments, the dissolution rate of the coated particle is at least 20% higher than the dissolution rate of the uncoated drug-containing core.

In some embodiments, the dissolution rate of the coated particle is at least 20% higher than the dissolution rate of the uncoated drug-containing core, wherein the solubility is measured by dissolving 120 mg of the coated or uncoated particles in water containing 0.75% Sodium lauryl sulfate (SLS) at room temperature for 30 minutes, with 75 RPM stirring.

In some embodiments, the coated particle has an improved flowability comparing to uncoated drug-containing core.

In some embodiments, the uncoated particles have a water contact angle that is between 90° and 145°.

In some embodiments, the uncoated particles have a water contact angle that is between greater than 145°.

In some embodiments, the coated particles have a water contact angle that is less than 900.

In one aspect, the disclosure is related to a method of preparing coated particles comprising active pharmaceutical ingredient (API)-containing core, a discontinuous zinc oxide coating layer and a discontinuous silicon oxide coating layer, the method comprising the sequential steps of:
(a) loading particles comprising an API into the chamber of a reactor, wherein the particles have a median particle size, on a volume average basis between 0.1 μm and 1000 μm;
(b1) applying vaporous or gaseous diethyl zinc to the particles in the reactor by pulsing the vaporous or gaseous diethyl zinc into the reactor;
(b2) performing one or more pump-purge cycles of the reactor using an inert gas;
(b3) applying vaporous or gaseous oxidant to the particles in the reactor by pulsing the oxidant into the reactor;
(b4) performing one or more pump-purge cycles of the reactor using an inert gas; (c)
repeating steps (b1)-(b4) at least once to create a discontinuous zinc oxide coating layer on the particles;
(e1) applying vaporous or gaseous silicon tetrachloride to the particles in the reactor by pulsing the vaporous or gaseous silicon precursor into the reactor;
(d2) performing one or more pump-purge cycles of the reactor using an inert gas;
(d3) applying vaporous or gaseous water to the particles in the reactor by pulsing vaporous or gaseous water into the reactor;
(d4) performing one or more pump-purge cycles of the reactor using an inert gas; and
(e) repeating steps (d1)-(d4) at least once to create a discontinuous silicon oxide coating layer on the discontinuous zinc oxide coating layer.

In some embodiments, the API is fenofibrate.

In some embodiments, the API is a BCS class II molecule or a class IV molecule.

In some embodiments, the uncoated particles have a water contact angle that is between 90° and 145°.

In some embodiments, the uncoated particles have a water contact angle that is between greater than 145°.

In some embodiments, the coated particles have a water contact angle that is less than 900.

In some embodiments, the zinc oxide coating is no more than 10 nm thick

In some embodiments, the silicon oxide layer is no more than 10 nm thick.

In some embodiments, the temperature of the chamber is maintained at a temperature between 20° C. and 60° C.

In some embodiments, the discontinuous coating layers, taken together do not provide a continuous pin-hole free coating of the API-containing core.

In some embodiments, the one or more inorganic coating layers, taken together constitute 2%-7% wt/wt of the coated particles.

In some embodiments, the one or more inorganic coating layers, taken together constitute 1%-3% wt/wt of the coated particles.

In some embodiments, the core has a median particle size, on a volume average basis, between 2 μm and 20 μm and the one or more inorganic coating layers, taken together constitute 2%-7% wt/wt of the coated particles.

In some embodiments, the core has a median particle size, on a volume average basis, between 0.1 μm and 1 μm and the one or more inorganic coating layers, taken together constitute 10%-20% wt/wt of the coated particles.

In some embodiments, the particles remain in the reactor during steps (b1)-(b4), each pump-purge cycle comprises flowing the inert gas into the reactor chamber to a desired pressure and after a delay time pumping the inert gas out of the reactor until the pressure of the inert gas is below 1 torr and repeating the steps of flowing the inert gas into the reactor chamber to a desired pressure and after a delay time pumping the inert gas out of the reactor until the pressure of the inert gas is below 1 torr.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a composition, “about” may mean+/−10% of the recited value. For instance, a composition including about 100 ng/ml of a given compound may include 90-110 ng/ml of the compound.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIGS. 1A-1B show the NMR spectrum of the coated (zinc oxide) and uncoated API (fenofibrate) particles.

FIG. 2 shows the XRD spectrum of the coated (zinc oxide) and uncoated API (fenofibrate) particles.

FIGS. 3A-3C show the TGA analysis results of the coated (zinc oxide) and uncoated API (fenofibrate) particles.

FIGS. 4A-4F show the SEM images of the coated (zinc oxide) and uncoated API (fenofibrate) particles. FIGS. 4A-4C show SEM images of uncoated API particles. FIGS. 4D-4F show SEM images of coated API particles.

FIG. 5 shows the relative percentage release over time in water containing 0.75% Sodium laureth sulfate (SLES) at room temperature, with a stirring of 75 revolutions per minute (RPM).

FIG. 6 shows the relative percentage release over time in water containing 0.75% Sodium laureth sulfate (SLES) at room temperature, with a stirring of 75 revolutions per minute (RPM). The WO-100C coated particles are formulated as 2 mg capsules. RLD is the commercial tablet formulation (120 mg tablets) of the reference listed drug (RLD) (a total tablet weight of 630 mg was used).

FIGS. 7A-7F show the SEM images of the coated (silicon oxide) and uncoated API (fenofibrate) particles. FIGS. 7A-7C show SEM images of uncoated API particles. FIGS. 7D-7F show SEM images of coated API particles.

FIG. 8 shows the relative percentage release over time in water containing 0.75% Sodium laureth sulfate (SLES) at room temperature, with a stirring of 75 revolutions per minute (RPM).

FIG. 9 shows the NMR spectrum of the coated and uncoated API particles. As shown in FIG. 9, there are no significant changes in NMR signals before and after the coating process. This result indicates that there is no structural change in the API after the zinc oxide plus silicon oxide coating, and that the API was not damaged by the coating process.

FIGS. 10A-10D show the SEM images of the coated API particles. The results indicate that there is no obvious change in particle size after the zinc oxide coating. Also, the SEM images show that the morphology of the coating is uncontinuous, patchy, and non-uniform (e.g., there are gaps, cracks and/or uncoated areas).

FIG. 11A-11C show the relative percentage release over time in water containing 0.75% Sodium laureth sulfate (SLS) at room temperature, with a stirring of 75 revolutions per minute (RPM). Fenofibrate with (1) zinc oxide coating and (2) zinc oxide plus silicon oxide coating showed a significant improvement in dissolution profile over uncoated particles. Further, for the tablet formulations, zinc oxide plus silicon oxide coating showed a significant improvement in dissolution profile over zinc oxide coating alone.

FIG. 12 shows the solubility in solutions containing different amounts of SLS at room temperature. As shown in FIG. 12, the solubility is largely the same for (1) uncoated particles, (2) zinc oxide coated particles and (2) zinc oxide plus silicon oxide coated particles.

FIGS. 13A-13B show the results of the pharmacokinetic studies of zinc oxide plus silicon oxide coated particles in Beagle dogs (n=6). FIG. 13A shows the mean serum concentration profile in the first 48 hours after injection. FIG. 13B is a zoom-in of FIG. 13A and shows the mean serum concentration profile in the first 8 hours after injection. Based on AUC, the zinc oxide plus silicon oxide coated particles showed a significant increase in bioavailability (~2 times) over the uncoated particles. The Cmax values indicated a significant increase (~3 times) over the uncoated particles. The Tmax values indicate quick availability of the API for absorption (i.e. resulting from improvement in dissolution rate).

FIG. 14 shows a schematic illustration of an exemplary reactor system.

FIG. 15 shows the relative percentage release over time in water containing 0.75% Sodium laureth sulfate (SLS) at room temperature, with a stirring of 75 revolutions per minute (RPM). Fenofibrate with (1) zinc oxide coating (coating 1) and (2) zinc oxide plus silicon oxide coating (coating 2) showed a significant improvement in dissolution profile over uncoated particles.

FIG. 16 shows the results of the pharmacokinetic studies of zinc oxide plus silicon oxide coated particles in Wister rats (n=6).

FIG. 17A-17B show the results from stability studies. FIG. 17A shows the results from uncoated fenofibrate particles. FIG. 17B shows the results from zinc oxide plus silicon oxide coated fenofibrate particles.

FIGS. 18A-18C show the results from toxicity studies. FIG. 18A shows the properties (e.g., physical appearance) of the coated and uncoated fenofibrate particles. FIG. 18B shows the study protocol and the results. FIG. 18C shows the histopathology images of major organs (including the liver, the lung, the kidney, and the heart) after administration of the coated and uncoated fenofibrate particles on the 29^{th} day.

### DETAILED DESCRIPTION

This disclosure pertains to a coated particle that has a API-containing core and an inorganic coating layer and methods of preparing thereof. The coated API-containing particle has an improved solubility and/or dissolution rate compared to uncoated API particles. The core comprises or consists of one or more (preferably) poorly soluble APIs. The API can be a BCS Class II molecule. In some cases the API is a BCS class IV molecule (low solubility and low permeability). In addition to one or more APIs, the core can contain one or more pharmaceutically acceptable excipients. In some cases, the core consists of one or more APIs.

### Drug

The term “drug,” in its broadest sense includes all small molecule or biological APIs that are organic molecules. The drug can be selected from the group consisting of an analgesic, an anesthetic, an anti-inflammatory agent, an anthelmintic, an anti-arrhythmic agent, an antiasthma agent, an antibiotic, an anticancer agent, an anticoagulant, an antidepressant, an antidiabetic agent, an antiepileptic, an antihistamine, an antitussive, an antihypertensive agent, an antimuscarinic agent, an antimycobacterial agent, an antineoplastic agent, an antioxidant agent, an antipyretic, an immunosuppressant, an immunostimulant, an antithyroid agent, an antiviral agent, an anxiolytic sedative, a hypnotic, a neuroleptic, an astringent, a bacteriostatic agent, a beta-adrenoceptor blocking agent, a blood product, a blood substitute, a bronchodilator, a buffering agent, a cardiac inotropic agent, a chemotherapeutic, a contrast media, a corticosteroid, a cough suppressant, an expectorant, a mucolytic, a diuretic, a dopaminergic, an antiparkinsonian agent, a free radical scavenging agent, a growth factor, a haemostatic, an immunological agent, a lipid regulating agent, a muscle relaxant, a parasympathomimetic, a parathyroid calcitonin, a biphosphonate, a prostaglandin, a radio-pharmaceutical, a hormone, a sex hormone, an anti-allergic agent, an appetite stimulant, an anoretic, a steroid, a sympathomimetic, a thyroid agent, a vaccine, a vasodilator and a xanthine.

Exemplary types of small molecule drugs include, but are not limited to, indomethacin, acetaminophen, clarithromycin, azithromycin, ibuprofen, fluticasone propionate, salmeterol, pazopanib HCl, palbociclib, and amoxicillin potassium clavulanate.

The drug can be a BCS Class II or BCS Class IV molecule. The drug can have a water solubility (solubility) of below 0.1 mg/ml, below 0.2 mg/ml, below 0.5 mg/ml, below 1 mg/ml, below 2 mg/ml, below 5 mg/ml, below 10 mg/ml, below 20 mg/ml, below 50 mg/ml, or below 100 mg/ml. The drug can have a solubility of above 0.05 mg/ml, above 0.1 mg/ml, above 0.2 mg/ml, above 0.5 mg/ml, above 1 mg/ml, above 2 mg/ml, above 5 mg/ml, above 10 mg/ml, above 20 mg/ml, above 50 mg/ml, or above 100 mg/ml. The drug can have a solubility of 0.1-100 mg/ml, 0.1-50 mg/ml, 0.1-20 mg/ml, 0.1-10 mg/ml, 0.1-5 mg/ml, 0.1-2 mg/ml, 0.1-1 mg/ml. The drug can have a solubility of below 1 mg/ml.

In some embodiments, the uncoated particles are poorly wettable. For example, the water contact angle on leveled particles is greater than 100, 120, 130, 140 or 1500 after 1 minute of contact. In some cases, the coated particles have a water contact angle on leveled particles that is less than 60, 50, 40, 30 or 200 after 1 minute of contact.

In some embodiments, the drug is in the form of uncoated particles. In some embodiments, uncoated particles have a surface area by BET (Brunauer, Emmett and Teller) of more than 0.1 m²/g, more than 0.2 m²/g, more than 0.5 m²/g, more than 1 m²/g, more than 2 m²/g, more than 5 m²/g, more than 10 m²/g, more than 20 m²/g, more than 50 m²/g, or more than 100 m²/g. In some embodiments, the uncoated particles have a surface area by BET of less than 0.1 m²/g, less than 0.2 m²/g, less than 0.5 m²/g, less than 1 m²/g, less than 2 m²/g, less than 5 m²/g, less than 10 m²/g, less than 20 m²/g, less than 50 m²/g, or less than 100 m²/g. In some embodiments, the uncoated particles have a surface area by BET of 0.1-100 m²/g, 0.1-50 m²/g, 0.1-20 m²/g, 0.1-10 m²/g, 0.2-10 m²/g, 0.5-10 m²/g, 1-10 m²/g, 2-10 m²/g, or 5-10 m²/g. In some embodiments, the uncoated particles have a surface area by BET of about 5.94 m²/g.

In some embodiments the uncoated particles have a median particle size, on a volume average basis, between 0.1 μm and 1000 μm. In some embodiments the uncoated particles have a median particle size, on a volume average basis, between 0.1 μm and 100 μm. In some embodiments the uncoated particles have a median particle size, on a volume average basis, between 0.1 μm and 50 μm. In some embodiments the uncoated particles have a median particle size, on a volume average basis, between 0.1 μm and 20 μm.

In some embodiments, the uncoated particles have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D10 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D10 of 0.1 μm to 200 μm, 0.1 μm to 1 μm, 0.1 μm to 10 μm, or 0.1 μm to 50 μm on a volume average basis. In some embodiments, the uncoated particles have a D10 of about 2 μm on a volume average basis.

In some embodiments, the uncoated particles have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D50 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D50 of about 4.5 μm on a volume average basis.

In some embodiments, the uncoated particles have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D90 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the coated particles have a D90 of 200 μm to 2000 μm on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D90 of about 9.2 μm on a volume average basis.

In some embodiments, the uncoated particles have a bulk density of below 0.05 mg/ml, 0.1 mg/ml, below 0.2 mg/ml, below 0.5 mg/ml, below 1 mg/ml, below 2 mg/ml, below 5 mg/ml, below 10 mg/ml, below 20 mg/ml, below 50 mg/ml, or below 100 mg/ml. In some embodiments, the uncoated particles have a bulk density of above 0.05 mg/ml, above 0.1 mg/ml, above 0.2 mg/ml, above 0.5 mg/ml, above 1 mg/ml, above 2 mg/ml, above 5 mg/ml, above 10 mg/ml, above 20 mg/ml, above 50 mg/ml, or above 100 mg/ml. In some embodiments, the uncoated particles have a bulk density of 0.1-100 mg/ml, 0.1-50 mg/ml, 0.1-20 mg/ml, 0.1-10 mg/ml, 0.1-5 mg/ml, 0.1-2 mg/ml, 0.1-1 mg/ml. In some embodiments, the uncoated particles have a bulk density of about 0.21 mg/ml.

In some embodiments, the uncoated particles have a tapped density of below 0.05 mg/ml, 0.1 mg/ml, below 0.2 mg/ml, below 0.5 mg/ml, below 1 mg/ml, below 2 mg/ml, below 5 mg/ml, below 10 mg/ml, below 20 mg/ml, below 50 mg/ml, or below 100 mg/ml. In some embodiments, the uncoated particles have a tapped density of above 0.05 mg/ml, above 0.1 mg/ml, above 0.2 mg/ml, above 0.5 mg/ml, above 1 mg/ml, above 2 mg/ml, above 5 mg/ml, above 10 mg/ml, above 20 mg/ml, above 50 mg/ml, or above 100 mg/ml. In some embodiments, the uncoated particles have a tapped density of 0.1-100 mg/ml, 0.1-50 mg/ml, 0.1-20 mg/ml, 0.1-10 mg/ml, 0.1-5 mg/ml, 0.1-2 mg/ml, 0.1-1 mg/ml. In some embodiments, the uncoated particles have a tapped density of about 0.49 mg/ml.

### Solubility/Dissolution

Poorly water-soluble drugs often require relatively high doses in order to reach therapeutic plasma concentrations after oral administration. While solubility is an equilibrium measurement, the dissolution rate of drug is important when dissolution is time limited. Various methods, including micronization and conversion to an amorphous form can be used to increase solubility and/or dissolution rate.

In some embodiments, the solubility is water solubility. In some embodiments, the solubility is assessed in water containing 0.75% Sodium laureth sulfate (SLS). In some embodiments, the solubility is assessed in water containing 0.75% SLS at room temperature, with a stirring of 75 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours, more than 48 hours, or more than 72 hours. In some embodiments, the solubility is assessed in PBS buffer. In some embodiments, the solubility is assessed in a buffer with physiological pH.

### BCS Class II Drugs

Biopharmaceutics classification system (BCS) is a scientific classification of a drug substance based on its aqueous solubility and intestinal permeability that correlates in vitro dissolution and in vivo bioavailability of drug products (Table 1).

TABLE 1

| BCS class | Solubility | Permeability | Examples |
|---|---|---|---|
| I | High | High | Metoprolol, Diltiazem, |
| | | | Propranolol |
| II | Low | High | Phenytoin, Nifedipine, |
| | | | Danazol |
| III | High | Low | Cimetidine, Acyclovir, |
| | | | Captopril |
| IV | Low | Low | Hydrochlorothiazide, Taxol, |
| | | | Furosemide |

A discussion of BCS Class II drugs can be found, e.g., in Kumar, Sumit, et al. “Drug carrier systems for solubility enhancement of BCS class II drugs: a critical review.” Critical Reviews™ in Therapeutic Drug Carrier Systems 30.3 (2013); Khadka, Prakash, et al. “Pharmaceutical particle technologies: An approach to improve drug solubility, dissolution and bioavailability.” Asian Journal of Pharmaceutical Sciences 9.6 (2014): 304-316; each of which is incorporated herein by reference in its entirety.

### Atomic Layer Coating (ALC)

In the vapor phase deposition method (also called atomic layer coating), a thin film coating is formed on at least a portion the surface of a particle by depositing successive atomic layers of one or more coating materials. In some embodiment, the coating material is zinc oxide. In some embodiment, the coating material is aluminum oxide. In some embodiments, the coating material is silicon oxide.

### Reactor System

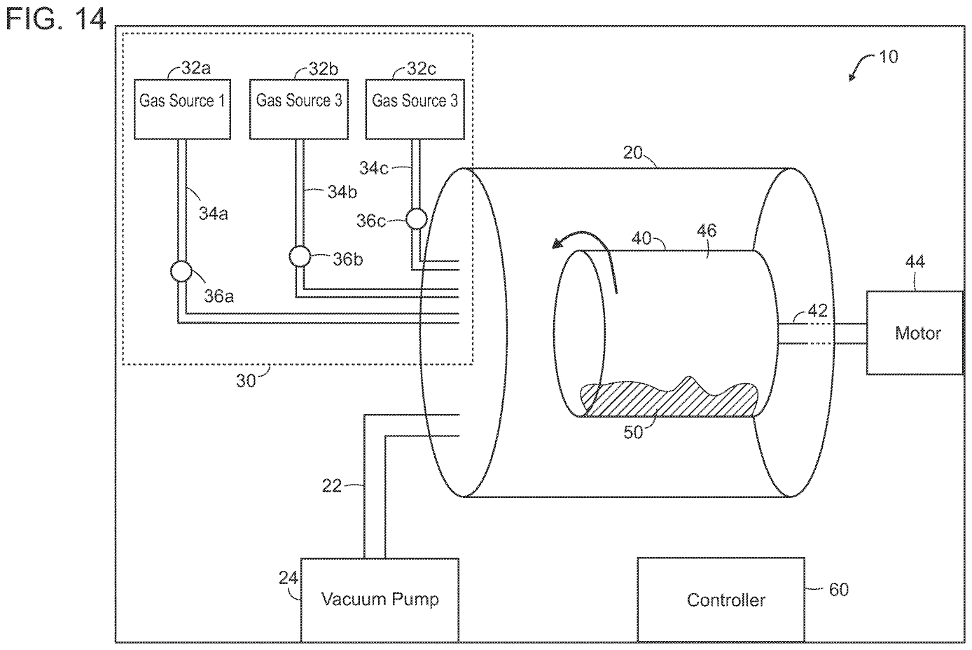

The term “reactor system” in its broadest sense includes all systems that could be used to perform ALC. An exemplary reactor system is illustrated in FIG. 14 and further described below.

FIG. 14 illustrates a reactor system 10 for performing coating of particles, with thin-film coatings. The reactor system 10 can perform ALC coating. The reactor system 10 permits ALC coating to be performed at higher (above 50° C., e.g., 50-100° C. or higher) or lower processing temperature, e.g., below 50° C., e.g., at or below 25° C. For example, the reactor system 10 can form thin-film metal metalloid oxide on the particles primarily by ALC at temperatures of 40-80° C., e.g., 40° C. or 80° C. In general, the particles can remain or be maintained at such temperatures. This can be achieved by having the reactants and/or the interior surfaces of the reactor chamber (e.g., the chamber 20 and drum 40 discussed below) remain or be maintained at such temperatures. One of the gas sources can be a zinc precursor. In particular, a gas source can provide a vaporous or gaseous zinc precursor. For example, the zinc precursor can be diethylzinc (DEZ). One of the gas sources can be a silicone precursor. In particular, a gas source can provide a vaporous or gaseous silicon precursor. For example, the silicon precursor can be SiCl4. One of the gas sources can be a titanium precursor. In particular, a gas source can provide a vaporous or gaseous titanium precursor. For example, the titanium precursor can be TiCl₄.

Again, illustrating an ALC process for deposition of zinc oxide (other metal oxides and metalloid oxides can be deposited in similar manner using an appropriate precursor and oxidant), the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the ALC thin-film coating process by introducing a gaseous oxidant and zinc precursor into the chamber 20. The gaseous oxidant and zinc precursor are spiked alternatively into the reactor. In addition, the ALC reaction can be performed at low temperature conditions, such as below 80° C., e.g., below 50° C., below 30° C., or below 25° C. In some embodiments, the operating temperature is 25° C.

The chamber 20 is also coupled to a chemical delivery system 30. The chemical delivery system 30 includes three or more gas sources 32a, 32b, 32c coupled by respective delivery tubes 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 14 illustrates separate gas lines extending in parallel to the chamber for each gas source, two or more of the gas lines could be joined, e.g., by one or more three-way valves, before the combined line reaches the chamber 20.

One of the gas sources can provide an oxidant. In particular, a gas source can provide a vaporous or gaseous oxidant. For example, the oxidant can be water. As another example, the oxidant can be water vapor.

One of the gas sources can be a zinc precursor. In particular, a gas source can provide a vaporous or gaseous zinc precursor. For example, the zinc precursor can be diethylzinc (DEZ). One of the gas sources can be a silicone precursor. In particular, a gas source can provide a vaporous or gaseous silicon precursor. For example, the silicon precursor can be SiCl4. One of the gas sources can be a titanium precursor. In particular, a gas source can provide a vaporous or gaseous titanium precursor. For example, the silicon precursor can be TiO₂.

One of the gas sources can provide a purge gas. In particular, the third gas source can provide a gas that is chemically inert to the oxidant and zinc precursor, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 10 μm.

In operation, one of the gasses flows into chamber 20 from the chemical delivery system 30 as the drum 40 rotates. A combination of pores (1-100 um), holes (0.1-10 mm), or large openings in the coating drum 40 serve to confine the particles in the coating drum 40 while allowing rapid delivery of precursor chemistry and the pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to keep them separate, ensuring a large surface area of the particles remains exposed. This permits fast, uniform interaction of the particle surface with the process gas.

In some implementations, one or more temperature control components are integrated into the drum 40 to permit control of the temperature of the drum 40. For example, a resistive heater, a thermoelectric cooler, or other component can be in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44, etc. The controller 60 can receive the recipe as computer-readable data (e.g., that is stored on a non-transitory computer readable medium).

The controller 60 and other computing device parts of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, particles are loaded into the drum 40 in the reactor system 10. The particles can be purely particles of a drug (or a combination of particles of a first drug and a second drug) or a mixture of particles of a drug (or a combination of particles of a first drug and a second drug) and particles of an excipient. In some cases, the particles are composed of one or more drugs (e.g., one of the drugs discussed above) and one or more excipients. Once any access ports are sealed, the controller 60 operates the reactor system 10 according to the recipe in order to form the thin-film zinc oxide on the particles.

In particular, the oxidant and the precursor can be alternately supplied to the chamber 20, with each step of supplying an oxidant or the zinc precursor followed by a purge cycle in which the inert gas is supplied to the chamber 20 to force out the excessive oxidant or zinc precursor and by-products used in the prior step. Moreover, one or more of the gases (zinc precursor gases and/or the inert gas and/or oxidant gas) can be supplied in pulses in which the chamber 20 is filled with the gas to a specified pressure, a holding time is permitted to pass, and the chamber is evacuated by the vacuum pump 24 before the next pulse commences.

In particular, the controller 60 can operate the reactor system 10 as follows.

In a zinc precursor half-cycle, while the motor 44 rotates the drum 40 to agitate the particles 50:

i) The gas distribution system 30 is operated to flow the zinc precursor gas, e.g., diethylzinc (DEZ), from the source 32a into the chamber 20 until a first specified pressure is achieved. The specified pressure can be 0.1 Torr to half of the saturation pressure of the zinc precursor gas.

ii) Flow of the zinc precursor is halted, and a specified holding time is permitted to pass, e.g., as measured by a timer in the controller. This permits the zinc precursor to flow through the particle bed in the drum 40 and react with the surface of the particles 50 inside the drum 40.

iii) The vacuum pump 50 evacuates the chamber 20, e.g., down to pressures below 1 Torr, e.g., to 1 to 100 mTorr, e.g., 50 mTorr.

Next, in a first purge cycle, while the motor 44 rotates the drum to agitate the particles 50:

iv) The gas distribution system 30 is operated to flow the inert gas, e.g., N₂, from the source 32c into the chamber 20 until a second specified pressure is achieved. The second specified pressure can be 1 to 100 Torr.

v) Flow of the inert gas is halted, and a specified delay time is permitted to pass, e.g., as measured by the timer in the controller. This permits the inert gas to flow through the pores in the drum 40 and diffuse through the particles 50 to displace the zinc precursor gas and any vaporous by-products.

vi) The vacuum pump 50 evacuates the chamber 20, e.g., down to pressures below 1 Torr, e.g., to 1 to 500 mTorr, e.g., 50 mTorr.

These steps (iv)-(vi) can be repeated a number of times set by the recipe, e.g., six to twenty times, e.g., sixteen times.

In a oxidant half-cycle, while the motor 44 rotates the drum 40 to agitate the particles 50:

vii) The gas distribution system 30 is operated to flow the oxidant, e.g., water vapor, from the source 32a into the chamber 20 until a third specified pressure is achieved. The third pressure can be 0.1 Torr to half of the saturation pressure of the oxidant gas.

viii) Flow of the oxidant is halted, and a specified holding time is permitted to pass, e.g., as measured by the timer in the controller. This permits the oxidant to flow through the pores in the drum 40 and react with the surface of the particles 50 inside the drum 40.

ix) The vacuum pump 50 evacuates the chamber 20, e.g., down to pressures below 1 Torr, e.g., to 1 to 500 mTorr, e.g., 50 mTorr.

Next, a second purge cycle is performed. This second purge cycle can be identical to the first purge cycle or can have a different number of repetitions of the steps (iv)-(vi) and/or different delay time and/or different pressure.

The cycle of the zinc precursor half-cycle, first purge cycle, oxidant half cycle and second purge cycle can be repeated a number of times set by the recipe, e.g., one to ten times.

As noted above, the coating process can be performed at low processing temperature, e.g., below 80° C., e.g., at or below 50° C., at or below 35° C., or at or below 25° C. In particular, the particles can remain or be maintained at such temperatures during all of steps (i)-(ix) noted above. In general, the temperature of the interior of the reactor chamber does not exceed 80° C. during of steps (i)-(ix). This can be achieved by having the oxidant gas, zinc precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

### Methods for Preparing Pharmaceutical Compositions Comprising Drugs Encapsulated by a Zinc Oxide

In one aspect, the disclosure provides methods for preparing a pharmaceutical composition comprising a drug-containing core enclosed by zinc oxide.

The first exemplary method includes the sequential steps of: (a) loading the particles comprising the drug into a reactor, (b) applying a vaporous or gaseous zinc precursor to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. In some embodiments of the first exemplary method, the sequential steps (b)-(e) are optionally repeated one or more times to increase the total thickness of the zinc oxide that encloses the solid core of the coated particles. In some embodiments, the reactor pressure is allowed to stabilize following step (a), step (b), and/or step (d). In some embodiments, the reactor contents are agitated prior to and/or during step (b), step (c), and/or step (e). In some embodiments, a subset of vapor or gaseous content is pumped out prior to step (c) and/or step (e).

The second exemplary method includes (e.g., consists of) the sequential steps of (a) loading the particles comprising the drug into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous zinc precursor, (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. In some embodiments of the second exemplary method, the sequential steps (b)-(m) are optionally repeated one or more times to increase the total thickness of the one or more zinc oxide materials that enclose the solid core of the coated particles.

Some embodiments provide a method of preparing a pharmaceutical composition comprising coated particles comprising an active pharmaceutical ingredient enclosed by zinc oxide, the method comprising the sequential steps of: (a) providing uncoated particles comprising an active pharmaceutical ingredient (API); (b) performing atomic layer coating to apply a zinc oxide layer to uncoated particles comprising an active pharmaceutical ingredient thereby preparing coated particles comprising an active pharmaceutical ingredient enclosed by zinc oxide; (c) processing the coated particles to prepare a pharmaceutical composition wherein the processing comprising combining the particles with one or more pharmaceutically acceptable (e.g., acceptable in an oral drug product) excipients; and (d) processing the pharmaceutical composition to form a drug product (e.g, a pill, tablet or capsule). In some cases, the drug product is an oral drug product.

In some embodiments, the uncoated particles are at least 50% wt/wt API. In some embodiments, the uncoated particles are at least 70%, 80%, 90%, 99% or 100% wt/wt API. In some cases, the API is crystalline.

In some embodiments, the uncoated particles have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D10 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D10 of 0.1 μm to 200 μm, 0.1 μm to 1 μm, 0.1 μm to 10 μm, or 0.1 μm to 50 μm on a volume average basis. In some embodiments, the uncoated particles have a D10 of about 2 μm on a volume average basis.

In some embodiments, the uncoated particles have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D50 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D50 of about 4.5 μm on a volume average basis.

In some embodiments, the uncoated particles have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D90 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the coated particles have a D90 of 200 μm to 2000 μm on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D90 of about 9.2 μm on a volume average basis.

In some embodiments, the zinc oxide coating is a continuous (pinhole-free) conformal coating. The zinc oxide coating can be porous. The zinc oxide coating can be discontinuous. The zinc oxide coating can be patchy. The zinc oxide coating can be non-uniform. The coated particles can have exposed areas. The zinc oxide coating can have different thicknesses at different locations on the same coated particle. The zinc oxide coating can be discontinuous, patchy, and non-uniform. The zinc oxide coating can have gaps and/or cracks. The coated particle can have uncoated areas. The zinc oxide coating can be thin (e.g., less than 2 nm, less than 3 nm, less than 4 nm, less than 5 nm, less than 10 nm, less than 15 nm). The zinc oxide coating can be thin and patchy. The zinc oxide coating can be thin and non-uniform. The zinc oxide coating can be thin and discontinuous. The zinc oxide coating can be thin and can have exposed areas. The zinc oxide coating can be amorphous. The zinc oxide coating can be crystalline.

The zinc oxide coating may facilitate the application of a second layer of silicon oxide coating. The zinc oxide coating may help a silicon precursor adhere to the drug particle. The zinc oxide coating may help reduce steric hinderance for a silicon precursor to adhere to the drug particle. The zinc oxide coating may make it easier to apply a second layer of silicon oxide coating.

In some embodiments, the step of performing atomic layer coating comprises: (b1) loading the particles comprising the drug into a reactor; (b2) applying a vaporous or gaseous zinc precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas. In some embodiments, steps (b2)-(b5) are performed two or more times to increase the total thickness of the zinc oxide layer before step (c) is performed.

In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 25° C. and 55° C. In some embodiments, step (b) takes place at a temperature of about 25° C. In some embodiments, step (c) comprises combining the coated particles with one or more pharmaceutically acceptable excipients.

In some embodiments, the zinc oxide layer has a thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the zinc oxide layer has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the zinc oxide layer has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the zinc oxide layer has a thickness of between 1 nm and 10 nm. In some embodiments, the zinc oxide layer has a thickness of between 1 nm and 5 nm.

Some embodiments provide a pharmaceutical composition comprising coated particles comprising an active pharmaceutical ingredient enclosed by zinc oxide, prepared by a method comprising the sequential steps of: (a) providing uncoated particles comprising an active pharmaceutical ingredient; (b) performing atomic layer coating to apply a zinc oxide layer to uncoated particles comprising an active pharmaceutical thereby preparing coated particles comprising an active pharmaceutical ingredient enclosed by zinc oxide; and (c) processing the coated particles to prepare a pharmaceutical composition.

In some embodiments, the step of performing atomic layer coating comprises:
(b1) loading the particles comprising the drug into a reactor; (b2) applying a vaporous or gaseous zinc precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas.

In some embodiments, steps (b2)-(b5) are performed two or more times to increase the total thickness of the zinc oxide layer before step (c) is performed. In some embodiments, the particles are agitated prior to and/or during step (a). In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 25° C. and 55° C. In some embodiments, step (b) takes place at a temperature of about 25° C.

In some embodiments, the zinc oxide layer has a thickness in range of 0.1 nm to 100 nm. In some embodiments, the uncoated particles have a median particle size, on a volume average basis between 0.1 μm and 1000 μm.

In some embodiments, the coated particles comprising an active pharmaceutical ingredient further comprise one or more pharmaceutically acceptable excipients. In some embodiments, the uncoated particles consist of the active pharmaceutical ingredient.

### Methods for Preparing Pharmaceutical Compositions Comprising Drugs Encapsulated by Silicon Oxide

In one aspect, the disclosure provides methods for preparing a pharmaceutical composition comprising a drug-containing core enclosed by silicon oxide.

In some embodiments, the silicon oxide coating is applied to the zinc oxide coated particle described herein. In some embodiments, the disclosure provides a coated particle comprising (1) a drug-containing core, (2) a zinc oxide coating layer, and (3) a silicon oxide coating layer. In some embodiments, the drug-containing core is first coated with a zinc oxide layer and then coated with a silicon oxide layer.

The first exemplary method includes the sequential steps of: (a) loading the particles comprising the drug into a reactor, (b) applying a vaporous or gaseous silicon precursor to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. In some embodiments of the first exemplary method, the sequential steps (b)-(e) are optionally repeated one or more times to increase the total thickness of the silicon oxide that enclose the solid core of the coated particles. In some embodiments, the reactor pressure is allowed to stabilize following step (a), step (b), and/or step (d). In some embodiments, the reactor contents are agitated prior to and/or during step (b), step (c), and/or step (e). In some embodiments, a subset of vapor or gaseous content is pumped out prior to step (c) and/or step (e).

The second exemplary method includes (e.g., consists of) the sequential steps of (a) loading the particles comprising the drug into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous silicon precursor, (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. In some embodiments of the second exemplary method, the sequential steps (b)-(m) are optionally repeated one or more times to increase the total thickness of the one or more silicon oxide materials that enclose the solid core of the coated particles.

Some embodiments provide a method of preparing a pharmaceutical composition comprising coated particles comprising an active pharmaceutical ingredient enclosed by silicon oxide, the method comprising the sequential steps of (a) providing uncoated particles comprising an active pharmaceutical ingredient (API); (b) performing atomic layer coating to apply a silicon oxide layer to uncoated particles comprising an active pharmaceutical ingredient thereby preparing coated particles comprising an active pharmaceutical ingredient enclosed by silicon oxide; (c) processing the coated particles to prepare a pharmaceutical composition wherein the processing comprising combining the particles with one or more pharmaceutically acceptable (e.g., acceptable in an oral drug product) excipients; and (d) processing the pharmaceutical composition to form a drug product (e.g, a pill, tablet or capsule). In some cases, the drug product is an oral drug product.

In some embodiments, the uncoated particles have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D10 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D10 of 0.1 μm to 200 μm, 0.1 μm to 1 μm, 0.1 μm to 10 μm, or 0.1 μm to 50 μm on a volume average basis. In some embodiments, the uncoated particles have a D10 of about 2 μm on a volume average basis.

In some embodiments, the uncoated particles have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D50 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D50 of about 4.5 μm on a volume average basis.

In some embodiments, the uncoated particles have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D90 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the coated particles have a D90 of 200 μm to 2000 μm on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D90 of about 9.2 μm on a volume average basis.

In some embodiments, the silicon oxide coating is a continuous (pinhole-free) conformal coating. The silicon oxide coating can be porous. The silicon oxide coating can be discontinuous. The silicon oxide coating can be patchy. The silicon oxide coating can be non-uniform. The coated particles can have exposed areas. The silicon oxide coating can have different thicknesses at different locations on the same coated particle. The silicon oxide coating can be discontinuous, patchy, and non-uniform. The silicon oxide coating can have gaps and/or cracks. The coated particle can have uncoated areas. The silicon oxide coating can be thin (e.g., less than 2 nm, less than 3 nm, less than 4 nm, less than 5 nm, less than 10 nm, less than 15 nm). The silicon oxide coating can be thin and patchy. The silicon oxide coating can be thin and non-uniform. The silicon oxide coating can be thin and discontinuous. The silicon oxide coating can be thin and can have exposed areas. The silicon oxide coating can be amorphous. In some embodiments, when water is used as the oxidant, the silicon oxide coating can be hydrophilic. In some embodiments, when ozone is used as the oxidant, the silicon oxide coating can be hydrophobic.

In some embodiments, the step of performing atomic layer coating comprises: (b1) loading the particles comprising the drug into a reactor; (b2) applying a vaporous or gaseous silicon precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas. In some embodiments, steps (b2)-(b5) are performed two or more times to increase the total thickness of the silicon oxide layer before step (c) is performed.

In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 25° C. and 55° C. In some embodiments, step (b) takes place at a temperature of about 25° C. In some embodiments, step (c) comprises combining the coated particles with one or more pharmaceutically acceptable excipients.

In some embodiments, the silicon oxide layer has a thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the silicon oxide layer has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the silicon oxide layer has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the silicon oxide layer has a thickness of between 1 nm and 10 nm. In some embodiments, the silicon oxide layer has a thickness of between 1 nm and 5 nm.

Some embodiments provide a pharmaceutical composition comprising coated particles comprising an active pharmaceutical ingredient enclosed by silicon oxide, prepared by a method comprising the sequential steps of: (a) providing uncoated particles comprising an active pharmaceutical ingredient; (b) performing atomic layer coating to apply a silicon oxide layer to uncoated particles comprising an active pharmaceutical thereby preparing coated particles comprising an active pharmaceutical ingredient enclosed by silicon oxide; and (c) processing the coated particles to prepare a pharmaceutical composition.

In some embodiments, the step of performing atomic layer coating comprises: (b1) loading the particles comprising the drug into a reactor; (b2) applying a vaporous or gaseous silicon precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas.

In some embodiments, steps (b2)-(b5) are performed two or more times to increase the total thickness of the silicon oxide layer before step (c) is performed. In some embodiments, the particles are agitated prior to and/or during step (a). In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 25° C. and 55° C. In some embodiments, step (b) takes place at a temperature of about 25° C.

In some embodiments, the silicon oxide layer has a thickness in range of 0.1 nm to 100 nm. In some embodiments, the uncoated particles have a median particle size, on a volume average basis between 0.1 μm and 1000 μm.

In some embodiments, the coated particles comprising an active pharmaceutical ingredient further comprise one or more pharmaceutically acceptable excipients. In some embodiments, the uncoated particles consist of the active pharmaceutical ingredient.

### Pharmaceutically Acceptable Excipients, Diluents, and Carriers

Pharmaceutically acceptable excipients include, but are not limited to:
(1) surfactants and polymers including: polyethylene glycol (PEG), polyvinylpyrrolidone (PVP), sodium lauryl sulfate, polyvinylalcohol, crospovidone, polyvinylpyrrolidone-polyvinylacrylate copolymer (PVPVA), cellulose derivatives, hydroxypropylmethyl cellulose, hydroxypropyl cellulose, carboxymethylethyl cellulose, hydroxypropyllmethyl cellulose phthalate, polyacrylates and polymethacrylates, urea, sugars, polyols, carbomer and their polymers, emulsifiers, sugar gum, starch, organic acids and their salts;
(2) binding agents such as cellulose, cross-linked polyvinylpyrrolidone, microcrystalline cellulose;
(3) filling agents such as lactose monohydrate, lactose anhydrous, microcrystalline cellulose and various starches;
(4) lubricating agents such as agents that act on the flowability of a powder to be compressed, including colloidal silicon dioxide, talc, stearic acid, magnesium stearate, calcium stearate, silica gel;
(5) sweeteners such as any natural or artificial sweetener including sucrose, xylitol, sodium saccharin, cyclamate, aspartame, and acesulfame K;
(6) flavoring agents;
(7) preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quaternary compounds such as benzalkonium chloride;
(8) buffers;
(9) diluents such as pharmaceutically acceptable inert fillers, such as microcrystalline cellulose, lactose, dibasic calcium phosphate, saccharides, and/or mixtures of any of the foregoing;
(10) wetting agents such as corn starch, potato starch, maize starch, and modified starches, and mixtures thereof;
(11) disintegrants such as croscarmellose sodium, crospovidone, sodium starch glycolate; and
(12) effervescent agents such as effervescent couples such as an organic acid (e.g., citric, tartaric, malic, fumaric, adipic, succinic, and alginic acids and anhydrides and acid salts), or a carbonate (e.g., sodium carbonate, potassium carbonate, magnesium carbonate, sodium glycine carbonate, L-lysine carbonate, and arginine carbonate) or bicarbonate (e.g. sodium bicarbonate or potassium bicarbonate).

### Coated Particles

In some embodiments, the disclosure provides coated particles comprising a drug-containing core and a zinc oxide coating layer. In some embodiments, the disclosure provides coated particles comprising a drug-containing core and a silicon oxide coating layer. In some embodiments, the disclosure provides coated particles comprising a drug-containing core, a zinc oxide coating layer, and a silicon oxide coating layer. In some embodiment, the drug is a BCS Class II molecule.

In some embodiments, the uncoated particles (drug-containing core) have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D10 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D10 of 0.1 μm to 200 μm, 0.1 μm to 1 μm, 0.1 μm to 10 μm, or 0.1 μm to 50 μm on a volume average basis. In some embodiments, the uncoated particles have a D10 of about 2 μm on a volume average basis.

In some embodiments, the uncoated particles have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D50 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D50 of about 4.5 μm on a volume average basis.

In some embodiments, the uncoated particles have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D90 of more than 0.1 μm, more than 0.2 μm, more than 0.5 μm, more than 1 μm, more than 2 μm, more than 5 μm, more than 10 μm, more than 20 μm, or more than 50 μm, on a volume average basis. In some embodiments, the coated particles have a D90 of 200 μm to 2000 μm on a volume average basis. In some embodiments, the uncoated particles have a D50 of 0.1 μm to 200 μm, 0.1 μm to 1 μm, 0.1 μm to 10 μm, or 0.1 μm to 50 μm on a volume average basis. In some embodiments, the uncoated particles have a D90 of about 9.2 μm on a volume average basis.

In some embodiments, the coated particles have an improved flowability comprising to uncoated drug-containing cores. In some embodiments, it is difficult to sieve the uncoated drug-containing cores through a 800 μm sieve. In some embodiments, the coated particles can be sieved through a 250 μm sieve with ease. In some embodiments, the coated particles can be sieved through a 105 μm sieve with ease.

In some embodiments, the API is not damaged by the coating process. In some embodiments, the structure of the API can be assessed by nuclear magnetic resonance (NMR) spectrum analysis. In some embodiments, there are no significant changes in NMR signals before and after the coating process. In some embodiments, there is no structural change in the API after the coating process, and that the API was not damaged by the coating process.

In some embodiments, the structure of the API can be assessed by—Ray Diffraction (XRD) analysis. In some embodiments, there are no significant changes in XRD signals before and after the coating process. In some embodiments, there is no structural change in the API after the coating process, and that the API was not damaged by the coating process.

In some embodiments, the composition of the coated particles can be assessed by Thermogravimetric Analysis (TGA %) analysis. In some embodiments, the amount of inorganic residue component constitutes more than 0.1%, more than 0.2%, more than 0.3%, more than 0.4%, more than 0.5%, more than 0.6%, more than 0.7%, more than 0.8%, more than 0.9%, more than 1%, more than 1.2%, more than 1.4%, more than 1.6%, more than 1.8%, more than 2%, more than 2.2%, more than 2.4%, more than 2.6%, more than 2.8%, more than 3%, more than 3.2%, more than 3.4%, more than 3.6%, more than 3.8%, more than 4%, more than 4.2%, more than 4.4%, more than 4.6%, more than 4.8%, more than 5%, more than 6%, more than 7%, more than 8%, more than 9%, more than 10%, more than 12%, more than 14%, more than 16%, more than 18%, or more than 20% wt/wt of the coated particles. In some embodiments, the amount of inorganic residue component constitutes less than 0.1%, less than 0.2%, less than 0.3%, less than 0.4%, less than 0.5%, less than 0.6%, less than 0.7%, less than 0.8%, less than 0.9%, less than 1%, less than 1.2%, less than 1.4%, less than 1.6%, less than 1.8%, less than 2%, less than 2.2%, less than 2.4%, less than 2.6%, less than 2.8%, less than 3%, less than 3.2%, less than 3.4%, less than 3.6%, less than 3.8%, less than 4%, less than 4.2%, less than 4.4%, less than 4.6%, less than 4.8%, less than 5%, less than 6%, less than 7%, less than 8%, less than 9%, less than 10%, less than 12%, less than 14%, less than 16%, less than 18%, or less than 20% wt/wt of the coated particles. In some embodiments, the amount of inorganic residue component constitutes 0.1%-20%, 0.5%-10%, 1%-10%, 1%-5%, 2%-5%, 1%-4%, 1%-3%, or 2%-4% wt/wt of the coated particles.

The morphology of the coated particles can be assessed by Scanning Electron Microscopy (SEM) analysis. There may be no obvious change in particle size before and after the coating process. There may be no obvious morphology change in the API before and after the coating process. The coating can be discontinuous. The coating can be patchy. The coating can be non-uniform. The coated particles can have exposed areas. The coating can have different thicknesses at different locations on the same coated particle. The coating can be discontinuous, patchy, and non-uniform. The coating can have gaps and/or cracks. The coated particle can have uncoated areas. The coating can be thin (e.g., less than 2 nm, less than 3 nm, less than 4 nm, less than 5 nm, less than 10 nm, less than 15 nm). The coating can be thin and patchy. The coating can be thin and non-uniform. The coating can be thin and discontinuous. The coating can be thin and can have exposed areas. The coating may reduce the surface charge of the coated particles, as compared to uncoated particles. The coating may help reduce agglomeration of the coated particles, as compared to uncoated particles. The coating may reduce the surface energy of the coated particles, as compared to uncoated particles. The coating may increase the wettability of the coated particles, as compared to uncoated particles. The coating may increase the hydrophilicity of the coated particles, as compared to uncoated particles. The coating may improve the flowability of the coated particles, as compared to uncoated particles. The coating may enhance the solubility of the coated particles, as compared to uncoated particles. The coating may enhance the dispersibility of the coated particles, as compared to uncoated particles. The coating may reduce the need for additional excipients in the final formulation.

In some embodiments, the coating comprises a thin zinc oxide coating layer and a thin silicon oxide coating layer. The total thickness of the coating can be 8-12 nm. The thickness of the zinc oxide coating layer may be 1-2 nm. The combination of an inner zinc oxide coating layer and an outer silicon oxide coating layer may further improve the dissolution rate of the coated particles, as compared to a zinc oxide coating layer alone.

In some embodiments, the dissolution of the coated particles can be assessed by an in vitro release overtime (dissolution) analysis. In some embodiments, the dissolution is assessed in water containing 0.75% Sodium laureth sulfate (SLS) at room temperature, with a stirring of 75 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours, more than 48 hours, or more than 72 hours. In some embodiments, the coated particles have an increased dissolution comparing to uncoated API particles. In some embodiments, the dissolution of the coated particles is at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, higher than the dissolution of uncoated particles (drug-containing cores).

In some embodiments, the coated particles are formulated into 2 mg capsules. In some embodiments, the dissolution of the coated particles (formulated as 2 mg capsules) can be assessed by an in vitro release over time (dissolution) analysis. In some embodiments, the dissolution is assessed in water containing 0.75% Sodium laureth sulfate (SLS) at room temperature, with a stirring of 75 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours, more than 48 hours, or more than 72 hours. In some embodiments, the coated particles (formulated as 2 mg capsules) have an increased dissolution comparing to uncoated API particles. In some embodiments, the dissolution of the coated particles (formulated as 2 mg capsules) is at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, higher than the dissolution of uncoated particles (drug-containing cores). In some embodiments, the dissolution of the coated particles (formulated as 2 mg capsules) is comparable to that of the commercial tablet formulation (120 mg tablets) of the reference listed drug (RLD). In some embodiments, the RLD tablet formulation is generated by melt dose technology (120 mg of API+>500 mg of excipients).

In some embodiments, the uncoated particles (drug-containing core) are coated by a pin-hole free zinc oxide coating. In some embodiments, the uncoated particles (drug-containing core) are coated by a pin-hole free silicon oxide coating. In some embodiments, the uncoated particles (drug-containing core) are coated by a zinc oxide coating that is not pin-hole free. In some embodiments, the uncoated particles (drug-containing core) are coated a silicon oxide coating that is not pin-hole free.

In some embodiments, a silicon oxide coating cannot be applied directly to the uncoated particles (drug-containing core) due to the surface properties of the drug. In some embodiments, a silicon oxide coating is not be applied directly to the uncoated particles (drug-containing core) due to the surface properties of the drug. In some embodiments, the uncoated particles (drug-containing core) is coated first by a zinc oxide coating before applying the silicon oxide coating. In some embodiments, the uncoated particles (drug-containing core) are coated first by a zinc oxide coating and then coated by a silicon oxide coating.

In some embodiments, an aluminum oxide coating cannot be applied directly to the uncoated particles (drug-containing core) due to the surface properties of the drug. In some embodiments, an aluminum oxide coating cannot be applied directly to the uncoated particles (drug-containing core) due to the surface properties of the drug. In some embodiments, the aluminum precursor trimethylaluminium (TMA) will react with the drug.

In some embodiments, a wet coating method cannot be used to coat the uncoated particles (drug-containing core) because the wet coating method may degrade/dissolve the uncoated particles (drug-containing core). In some embodiments, a dry coating method (e.g., ALC) is advantageous because it retains the integrity of the uncoated particles (drug-containing core).

In some embodiments, the bioavailability of the drug is limited by the surface characteristics (e.g., wettability and/or dispersibility) of the uncoated particles (drug containing core). In some embodiments, the coating (e.g., zinc oxide coating and/or silicon oxide coating) described herein can improve the surface characteristics of the uncoated particles (drug containing core). In some embodiments, the bioavailability of the drug is further improved by adding excipients (e.g., surfactants) to the drug formulation. In some embodiments, adding a zinc oxide coating can improve the wettability and/or dispersibility of the uncoated particles (drug containing core). In some embodiments, adding a zinc oxide coating can improve the dispersibility, but not the wettability of the uncoated particles (drug containing core). In some embodiments, adding a silicon oxide coating can improve the wettability and/or dispersibility of the uncoated particles (drug containing core). In some embodiments, adding a silicon oxide coating can improve the wettability, but not the dispersibility of the uncoated particles (drug containing core).

In some embodiments, wettability can be measured by measuring the contact angle. In some embodiments, a lower contact angle (×90°) signifies greater wettability, whereas a higher contact angle (≥90°) infers lower wettability. In some embodiments, the wettability of the coated particles are at least more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500% higher, comparing to uncoated particles (drug containing core). In some embodiments, the wettability of the coated particles are at least more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500% lower, comparing to uncoated particles (drug containing core).

In some embodiments, dispersibility in water can be measured by measuring the zeta potential of particle suspensions. In some embodiments, dispersibility in water can be measured by particle size distributions in water, as measured by laser diffraction. In some embodiments, the dispersibility of the coated particles are at least more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500% higher, comparing to uncoated particles (drug containing core). In some embodiments, the dispersibility of the coated particles are at least more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500% lower, comparing to uncoated particles (drug containing core).

In some embodiments, administering of the coated particles lead to improved drug bioavailability comparing to uncoated particles (drug containing core). In some embodiments, bioavailability can be determined by administering the coated particles and uncoated particles to beagle dogs. In some embodiments, bioavailability can be determined by measuring the maximum (or peak) serum concentration that the drug achieves (Cmax). In some embodiments, the drug bioavailability from the coated particles is at least more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500% higher, comparing to uncoated particles (drug containing core). In some embodiments, the drug bioavailability from the coated particles is at least more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500% lower, comparing to uncoated particles (drug containing core).

### Pharmaceutical Compositions

Also provided herein are pharmaceutical compositions that contain the coated particles. The pharmaceutical compositions can be formulated in any suitable manner known in the art. In some embodiments, the pharmaceutical compositions can be in the form of tablets, capsules, powders, microparticles, granules, syrups, suspensions, solutions, nasal spray, transdermal patches, injectable solutions, or suppositories.

Pharmaceutical compositions are formulated to be compatible with their intended route of administration (e.g., oral, intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents (e.g., benzyl alcohol or methyl parabens, chlorobutanol, phenol, ascorbic acid, and thimerosal), antioxidants (e.g., ascorbic acid and sodium bisulfite), chelating agents (e.g., ethylenediaminetetraacetic acid), buffers (e.g., acetates, citrates, and phosphates), and isotonic agents (e.g., sugars (e.g., dextrose), polyalcohols (e.g., mannitol or sorbitol), and salts (e.g., sodium chloride)), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating (e.g., lecithin) or a surfactant. Controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Pharmaceutically acceptable carriers, adjuvants and vehicles that can be used in the pharmaceutical compositions of the present disclosure include ion exchangers, alumina, aluminum stearate, lecithin, serum proteins (e.g., human serum albumin), buffer substances (e.g., phosphates, glycine), sorbic acid, potassium sorbate, partial glyceride mixtures of saturated vegetable fatty acids, water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms can contain the coated particles described herein in the range of 0.001% to 100% (e.g., 0.1-95%, 20-80%, or 75-85%) with the balance made up from the suitable pharmaceutically acceptable excipients.

Toxicity and therapeutic efficacy of compositions can be determined by standard pharmaceutical procedures in cell cultures or experimental animals (e.g., monkeys). One can, for example, determine the LD50 (the dose lethal to 50% of the population), the ED50 (the dose therapeutically effective in 50% of the population), and the therapeutic index (i.e., the ratio of LD50:ED50). Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given therapeutic agent for use in a subject (e.g., a human).

### EXAMPLES

The invention is further described in the following examples, which do not limit the scope of the invention described in the claims.

### Example 1: Zinc Oxide Coating

In step (a), ~30 grams of API (Fenofirate) were loaded to the rotatory reactor (rotating at 10-100 rpm). The rotatory reactor is beneficial because it can better expose the API particles. In step (b), diethyl zinc (DEZ) was pulsed into the reactor at about 40 torr and a reaction temperature of 22° C. In step (c), the reactor was purged using an inert gas to remove excess DEZ. In step (d), oxidant (e.g., water) was pulsed into the reactor with a reaction temperature of 22° C. In step (e), the reactor was purged using an inert gas to remove excess water vapor. Steps (b)-(e) were repeated to achieve the desired coating thickness.

Three different zinc oxide coating thickness was applied (by varying the cycle numbers). Based on the thickness of coating, The coated fenofibrate particles are named WO-100A, WO-100B, and WO-100C. The specific coating parameters are shown in the table below.

TABLE 2
Zinc Oxide Coating Parameters

| | Sample | | | |
|---|---|---|---|---|
| Sample ID | Load (g) | T (° C.) | No. of cycles | μdose of DEZ/H2O |
| WO-100A | 30 | 22 | 15 | 2/2 |
| WO-100B | 27 | 22 | 50 | 2/2 |
| WO-100C | 24 | 22 | 75 | 2/2 |

### Example 2: Analysis of the Zinc Oxide Coated Particles

To evaluate if encapsulation of API by zinc oxide coatings altered the structure of the API or changed the properties of the API particles, the coated particles were subjected to various analysis to determine if the zinc oxide coatings altered the structure of the API or the properties of the API particles.

### Nuclear Magnetic Resonance (NMR) Analysis

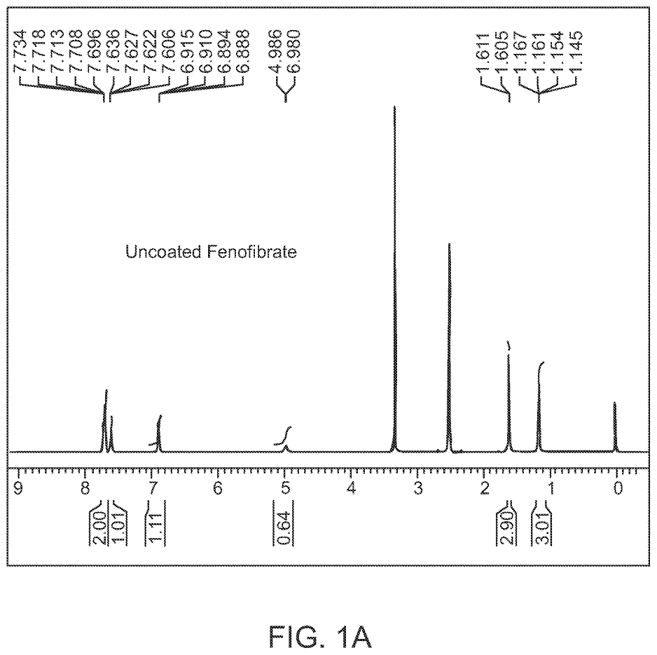

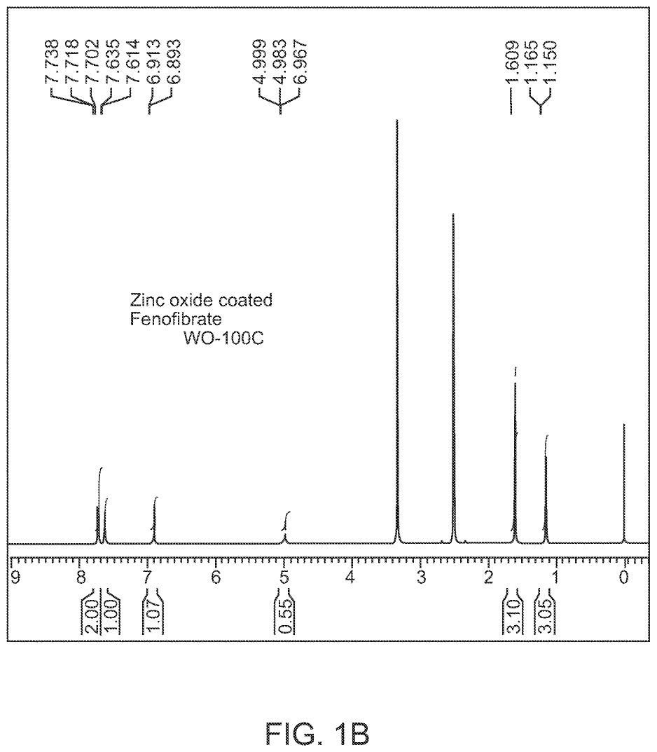

FIGS. 1A-1B show the NMR spectrum of the coated and uncoated API particles. As shown in FIGS. 1A-1B, there are no significant changes in NMR signals before and after the coating process. This result indicates that there is no structural change in the API after the zinc oxide coating, and that the API was not damaged by the coating process.

### X-Ray Diffraction (XRD) Analysis

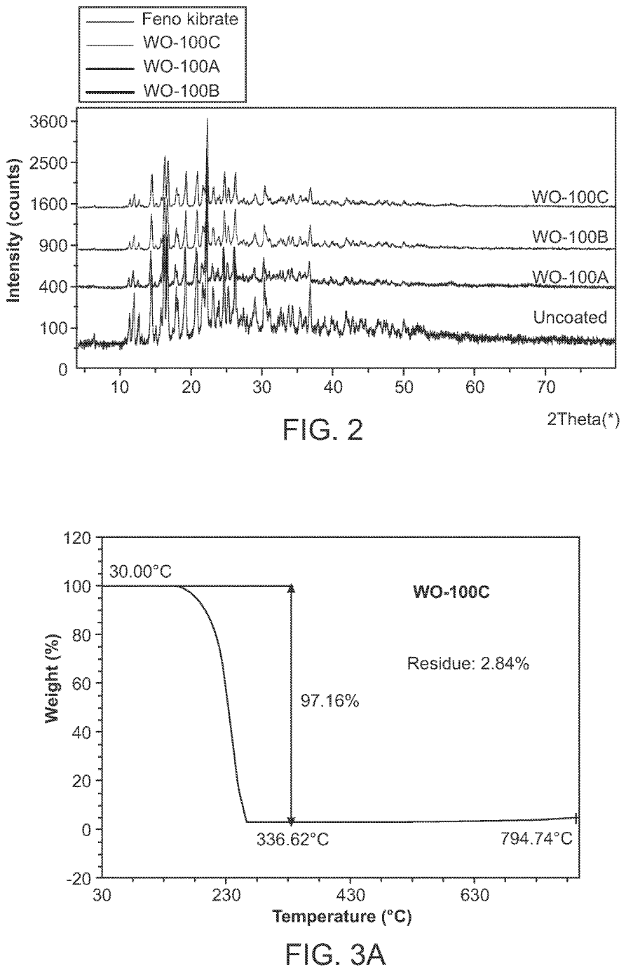

FIG. 2 shows the XRD spectrum of the coated and uncoated API particles. As shown in FIG. 2, there are no significant changes in XRD signals before and after the coating process. This result indicates that there is no phase change after the zinc oxide coating, and that the API was not damaged by the coating process.

### Thermogravimetric Analysis (TGA %) Analysis

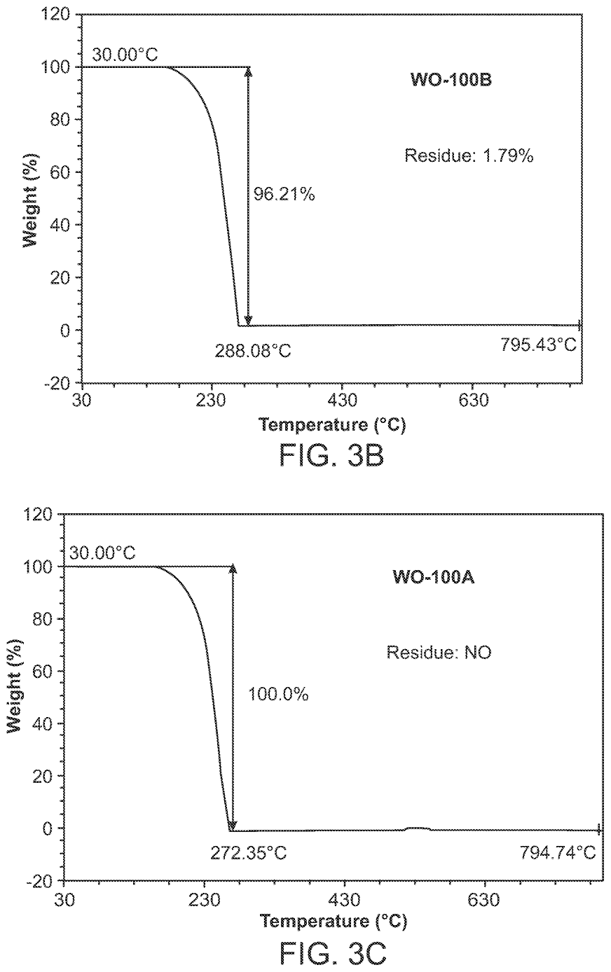

FIGS. 3A-3C show the TGA analysis results of the coated and uncoated API particles. As shown in FIG. 3A, the residue inorganic material in WO-100C was about 2.84% wt/wt. As shown in FIG. 3B, the residue inorganic material in WO-100B was about 1.79% wt/wt. As shown in FIG. 3C, the residue inorganic material in WO-100A was about 0% wt/wt.

### Scanning Electron Microscopy (SEM) Analysis

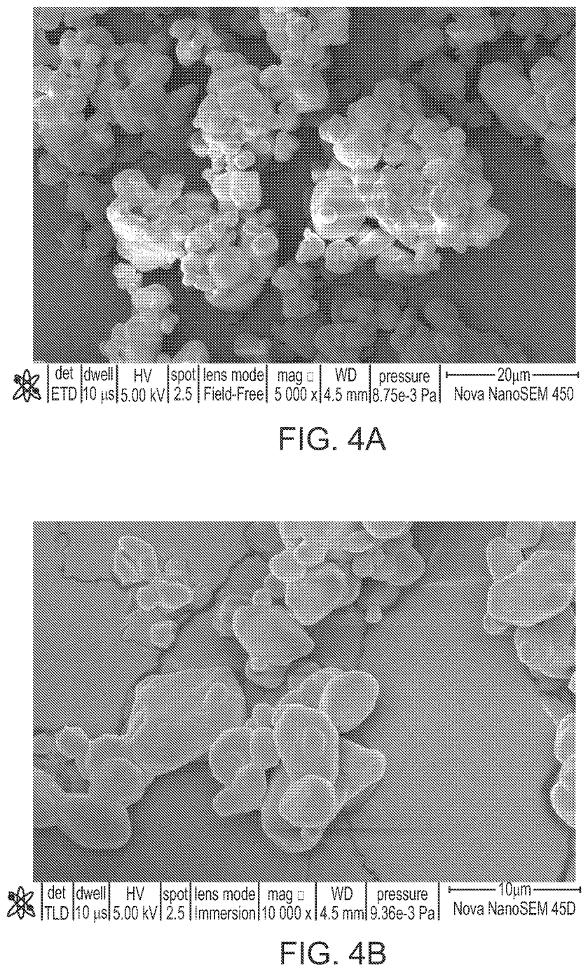

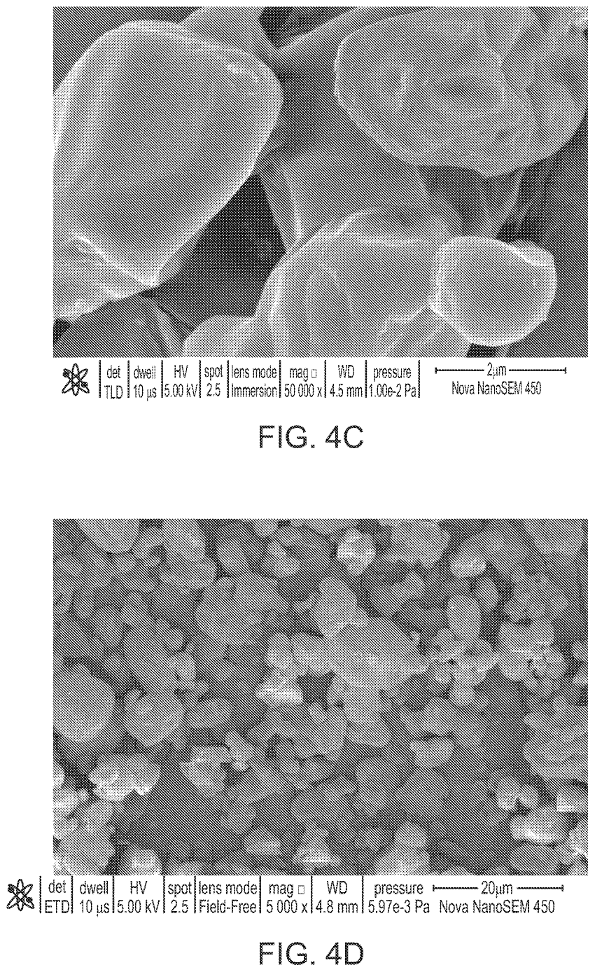

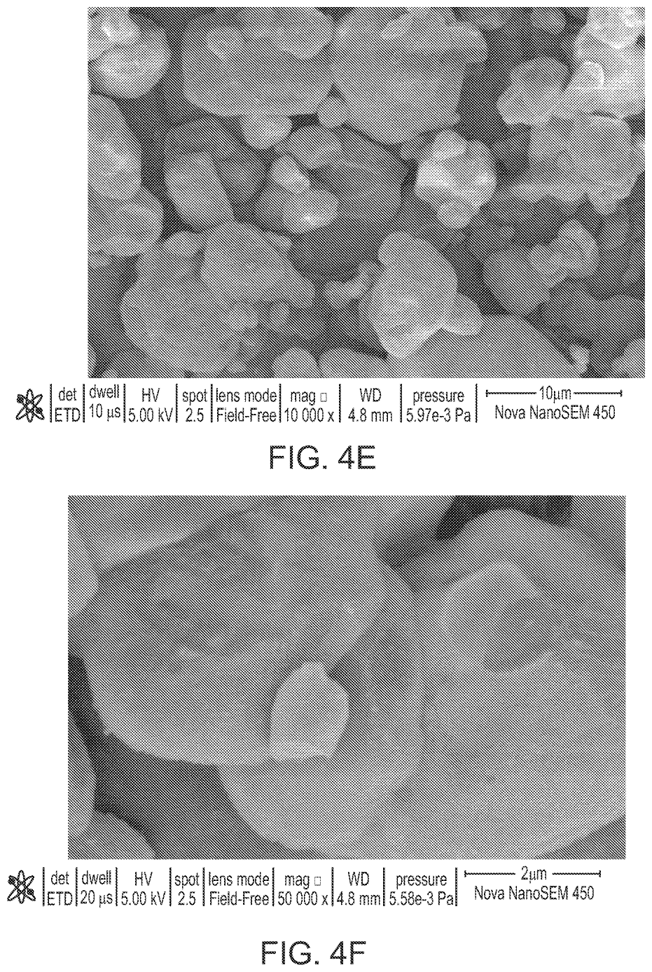

FIGS. 4A-4F show the SEM images of the coated and uncoated API particles. FIGS. 4A-4C show SEM images of uncoated API particles. FIGS. 4D-4F show SEM images of coated API particles. The results indicate that there is no obvious change in particle size after the zinc oxide coating. The results also indicate that the coating is discontinuous.

### Release Over Time (Dissolution) Analysis

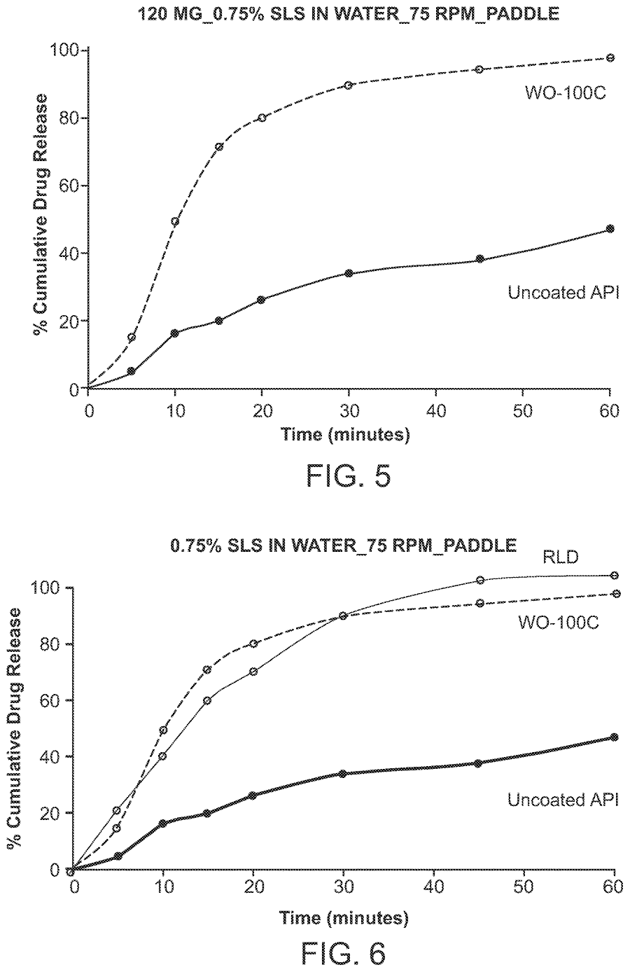

FIG. 5 shows the relative percentage release over time in water containing 0.75% Sodium laureth sulfate (SLS) at room temperature, with a stirring of 75 revolutions per minute (RPM). The results showed a significant improvement in dissolution profile of fenofibrate with 2 to 5 wt. % of zinc oxide coating. Specifically, after the zinc oxide coating, the cumulative drug release in water containing 0.75% SLS increased by about 100% after 30 minutes.

FIG. 6 shows the relative percentage release over time in water containing 0.75% Sodium laureth sulfate (SLS) at room temperature, with a stirring of 75 revolutions per minute (RPM). The WO-100C coated particles are formulated as 2 mg capsules. RLD is the commercial tablet formulation (120 mg tablets) of the reference listed drug (RLD) (a total tablet weight of 630 mg was used). The RLD tablet formulation is generated by melt dose technology (120 mg of API+>500 mg of excipients). The results indicate that zinc oxide coated API shows similar dissolution performance as RLD without the aid of any additional excipients, thus significantly lowering the pill burden.

### Example 3: Silicon Oxide Coating

To further assess the effects of silicon oxide coating on dissolution, the API (Fenofibrate) particles were coated with a layer of silicon oxide.

In step (a), about 30-60 grams API (Fenofibrate) particles were loaded to the rotatory reactor (rotating at 10-100 rpm). In step (b), Silicon tetrachloride (SiCl4) was pulsed into the reactor at about 40 torr and a reaction temperature of 25° C. In step (c), the reactor was purged using an inert gas to remove excess SiCl4. In step (d), water was pulsed into the reactor with a reaction temperature of 25° C. In step (e), the reactor was purged using an inert gas to remove excess water vapor. Steps (b)-(e) were repeated to achieve the desired coating thickness.

Four different silicon oxide coating thickness was applied (by varying the cycle numbers). Based on the thickness of coating, The coated fenofibrate particles are named WO-101A, WO-101B, WO-101C and WO-101D. The specific coating parameters are shown in the table below.

TABLE 3
Silicon Oxide Coating Parameters

| | | | Precursor | Hold | | |
|---|---|---|---|---|---|---|
| | | | Pressure | time | | |
| | Sample | | (SiCl₄/ | (TMA/ | | microdose |
| Sample | Load | T | H₂O) | H₂O) | No. of | of |
| ID | (g) | (° C.) | (Torr.) | (s) | cycles | SiCl₄/H2O |
| WO-101A | 30 | 25 | 40/8 | 120/60 | 30 | 1/2 |
| WO-101B | 28 | 25 | 40/8 | 120/60 | 60 | 1/2 |
| WO-101C | 27 | 25 | 40/8 | 120/60 | 113 | 1/2 |
| WO-101D | 26 | 25 | 40/8 | 120/60 | 143 | 1/2 |

### Example 4: Analysis of the Silicon Oxide Coated Particles

To evaluate if encapsulation of API by silicon oxide coatings altered the structure of the API or changed the properties of the API particles, the coated particles were subjected to various analysis to determine if the silicon oxide coatings altered the structure of the API or the properties of the API particles.

### Scanning Electron Microscopy (SEM) Analysis

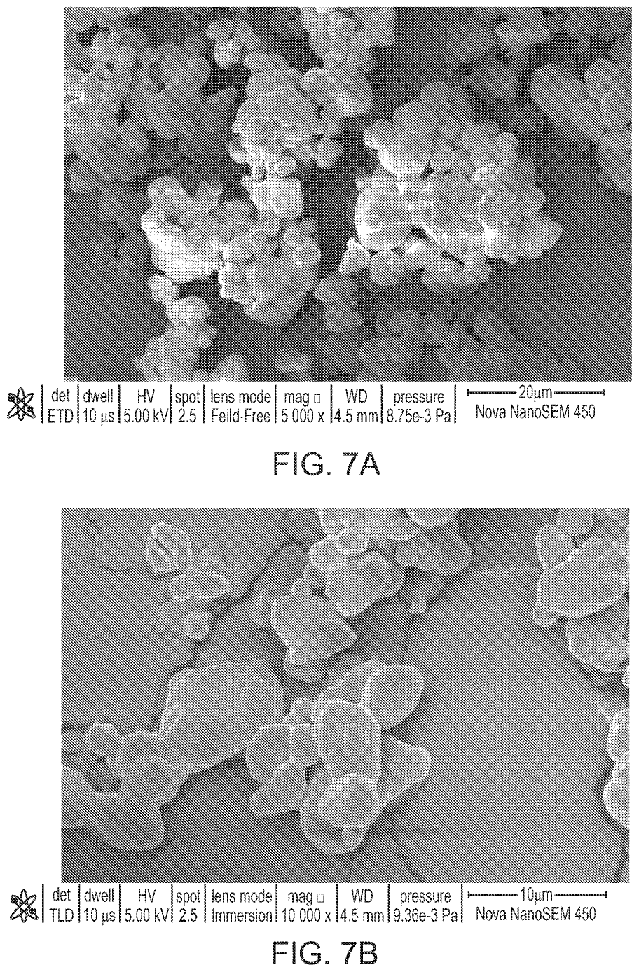

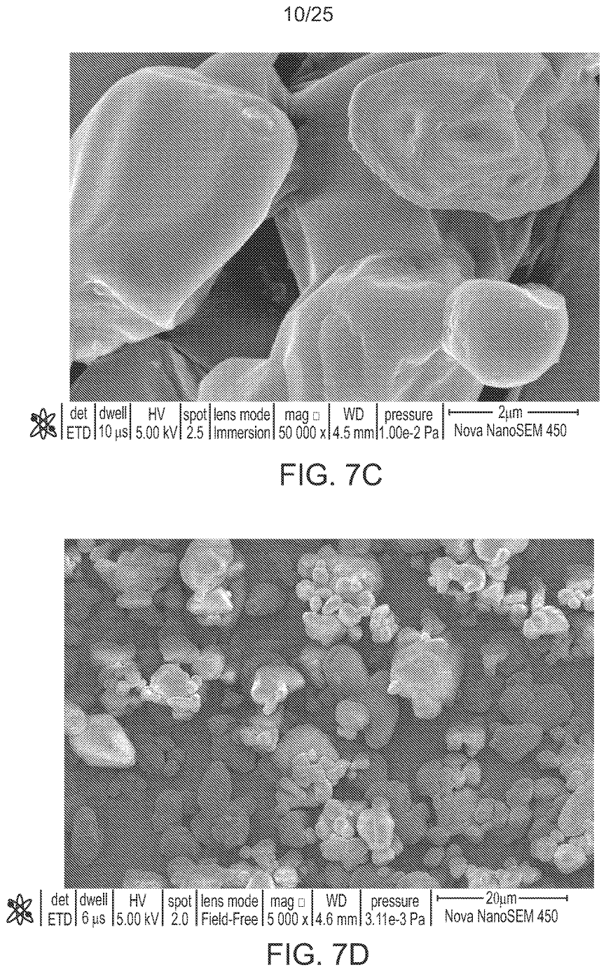

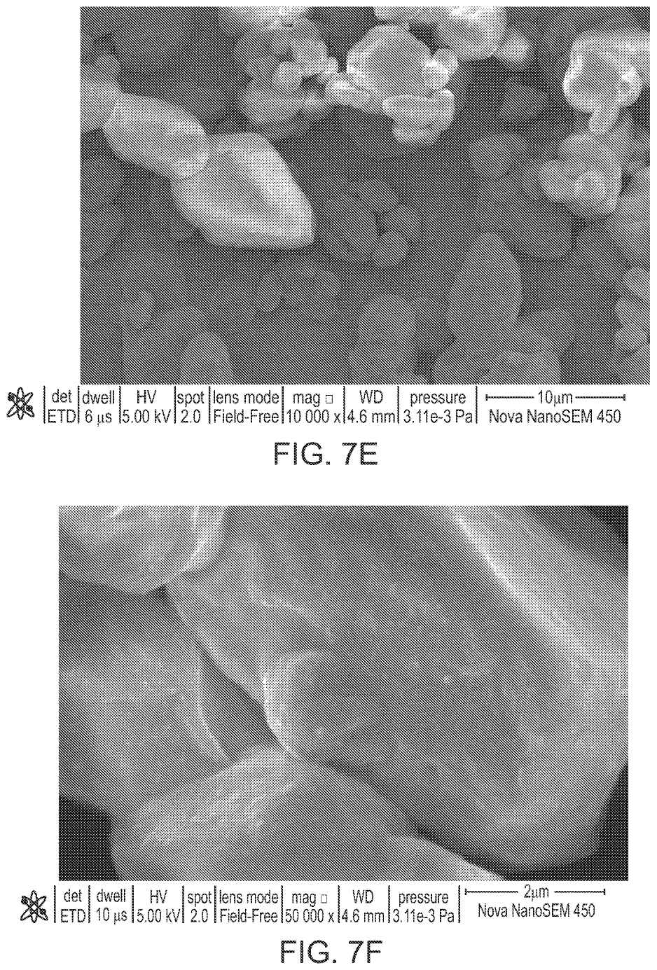

FIGS. 7A-7F show the SEM images of the coated and uncoated API particles. FIGS. 7A-7C show SEM images of uncoated API particles. FIGS. 7D-7F show SEM images of coated API particles. The results indicate that there is no obvious change in particle size after the silicon oxide coating. The results indicate the coating is discontinuous.

### Release Over Time (Dissolution) Analysis

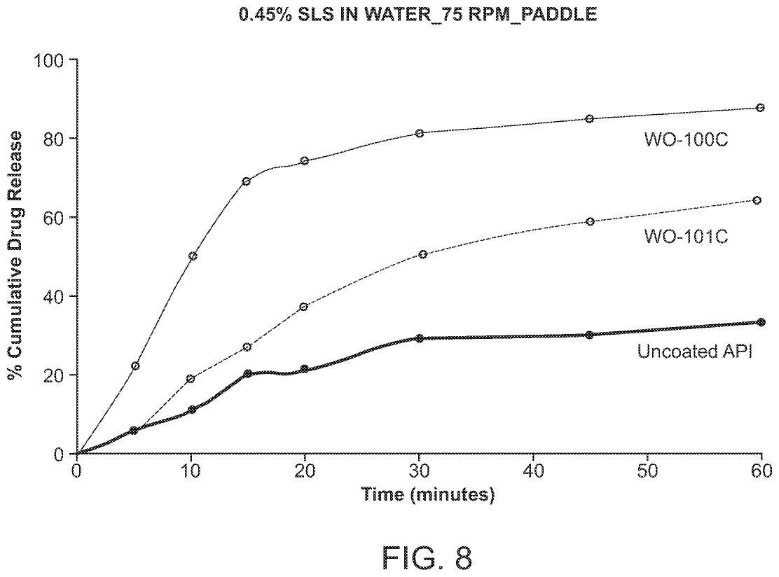

FIG. 8 shows the relative percentage release over time in water containing 0.75% Sodium laureth sulfate (SLS) at room temperature, with a stirring of 75 revolutions per minute (RPM). Fenofibrate with silicon oxide coating (WO-101C) showed a significant improvement in dissolution profile over uncoated particles. Specifically, after the silicon oxide coating, the cumulative drug release in water containing 0.75% SLS increased by about 100% after 60 minutes, as compared to that of uncoated particles.

### Example 5: Zinc Oxide Coating and Silicon Oxide Coating

Further, silicon oxide coating was applied on top of zinc oxide coating to create zinc oxide plus silicon oxide coated particles.

Specifically, either zinc oxide coating alone or zinc oxide plus silicon oxide coating (applying silicon oxide coating on top of zinc oxide coating) were applied to obtain (1) zinc oxide coated particles (WO-1101B and WO-115A), and (2) zinc oxide plus silicon oxide coated particles (WO-114 and WO-1115). The specific coating parameters are shown in the table below.

TABLE 4
Zinc oxide coating and zinc oxide plus
silicon oxide coating parameters

| | | | | | TGA | |
|---|---|---|---|---|---|---|
| | Sample | | | | (wt. %) | ICP- |
| Sample | Load | T | | No. of | GPC | OES |
| ID | (g) | (° C.) | Precursors | cycles | (nm/cycle) | (Zn %) |
| WO-110B | 57 | 24 | DEZ/H2O | 50 | 0.8 | 0.6 |
| WO-114 | 55 | 24 | SiCl₄/H₂O | 45 | 3 | 0.6 |
| (WO-110B) | | | | | | (0.83 |
| | | | | | | for Si) |
| WO-115A | 57 | 24 | DEZ/H2O | 50 | 0.8 | 0.6 |
| WO-115B | 55 | 24 | SiCl₄/H₂O | 45 | 3 | 0.6 |
| | | | | | | (0.83 |
| | | | | | | for Si) |

### Thermogravimetric Analysis (TGA %) Analysis

Table 4 shows the TGA analysis results of the coated API particles. As shown in Table 4, the zinc oxide coated particles (WO-110B and WO-115A) have about 0.8 wt % inorganic material (zinc oxide coating). The zinc oxide plus silicon oxide coated particles (WO-114 and WO-115B) have about 3 wt % inorganic material (zinc oxide plus silicon oxide coating).

### Nuclear Magnetic Resonance (NMR) Analysis

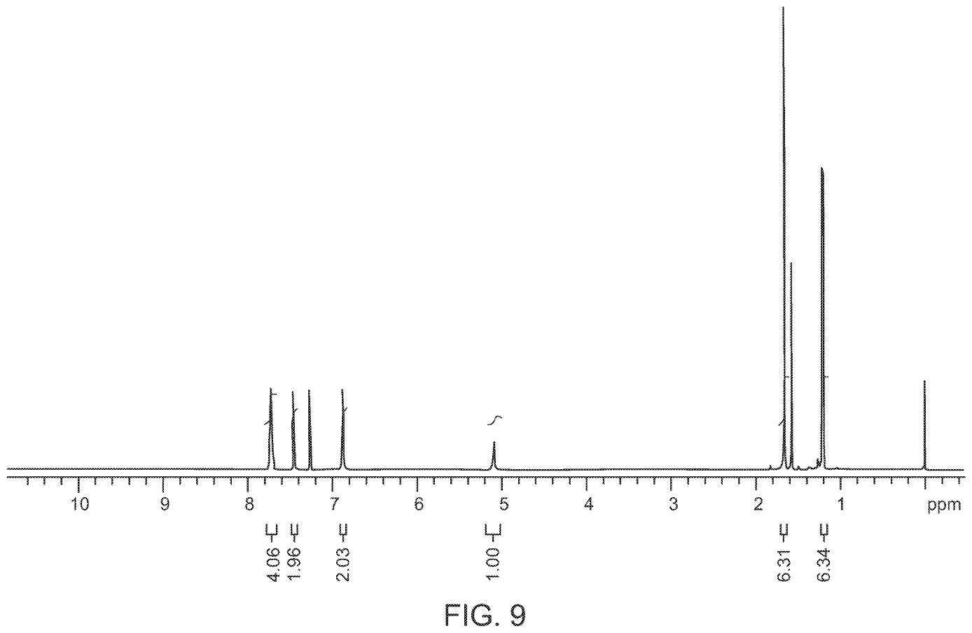

FIG. 9 shows the NMR spectrum of the coated and uncoated API particles. As shown in FIG. 9, there are no significant changes in NMR signals before and after the coating process. This result indicates that there is no structural change in the API after the zinc oxide plus silicon oxide coating, and that the API was not damaged by the coating process.

### Scanning Electron Microscopy (SEM) Analysis

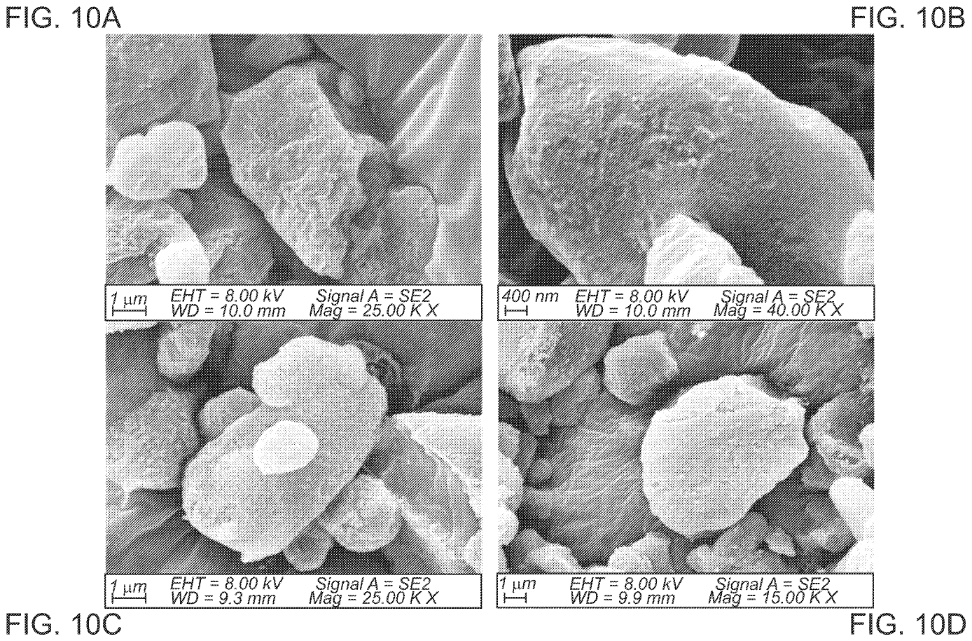

FIGS. 10A-10D show the SEM images of the coated API particles. The results indicate that there is no obvious change in particle size after the zinc oxide coating. Also, the SEM images show that the morphology of the coating is discontinuous, patchy, and non-uniform (e.g., there are gaps, cracks and/or uncoated areas).

### Release Over Time (Dissolution) Analysis

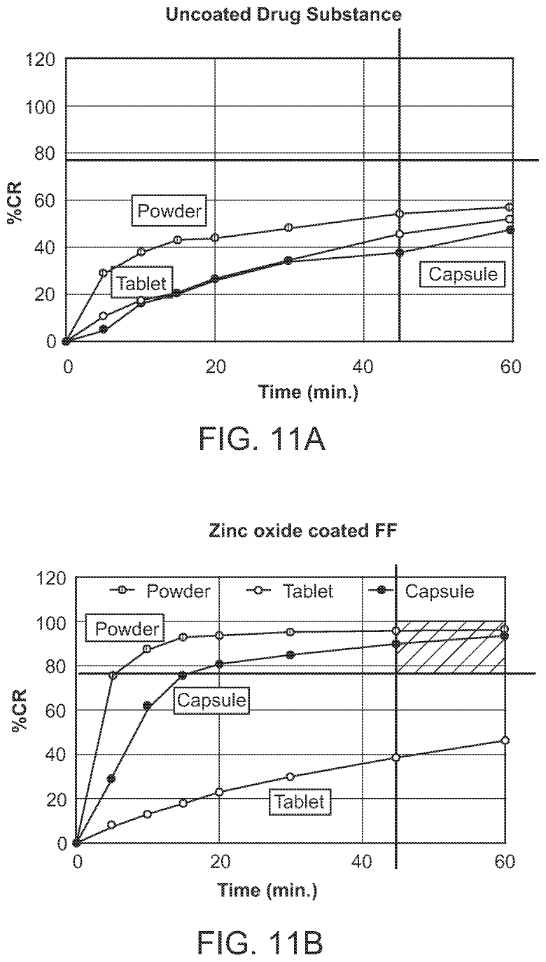

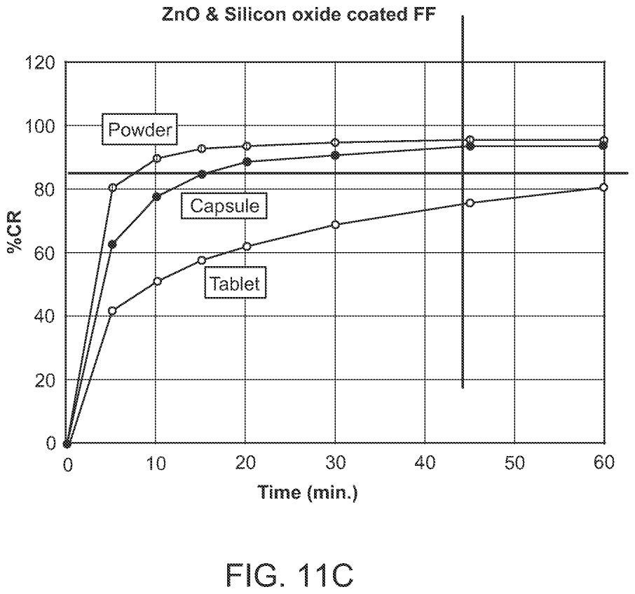

FIG. 11A-11C shows the relative percentage release over time in water containing 0.75% Sodium laureth sulfate (SLS) at room temperature, with a stirring of 75 revolutions per minute (RPM). Fenofibrate with (1) zinc oxide coating and (2) zinc oxide plus silicon oxide coating showed a significant improvement in dissolution profile over uncoated particles. Further, for the tablet formulations, zinc oxide plus silicon oxide coating showed a significant improvement in dissolution profile over zinc oxide coating alone.

Dissolution data for coated particles shows significant improvement against the native API in powder form and capsule form.

Advantage of dispersibility is lost in the tableting process for zinc oxide coated particles.

Solubility data also suggest that the improvement in dissolution is likely be due to better dispersibility of powder over liquid.

Zinc oxide plus silicon oxide coated particles tablet shows better release compared to zinc oxide coated particles tablet, but still lesser than the powder and capsule form.

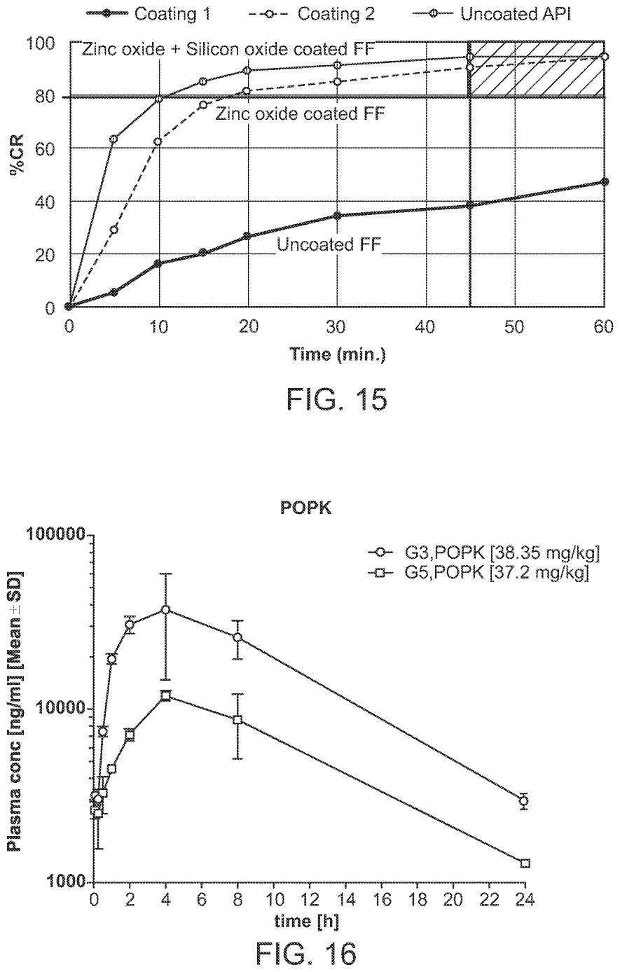

FIG. 15 shows the relative percentage release over time in water containing 0.75% Sodium laureth sulfate (SLS) at room temperature, with a stirring of 75 revolutions per minute (RPM). Fenofibrate with (1) zinc oxide coating (coating 1) and (2) zinc oxide plus silicon oxide coating (coating 2) showed a significant improvement in dissolution profile over uncoated particles.

### Solubility Analysis in the Presence of a Detergent

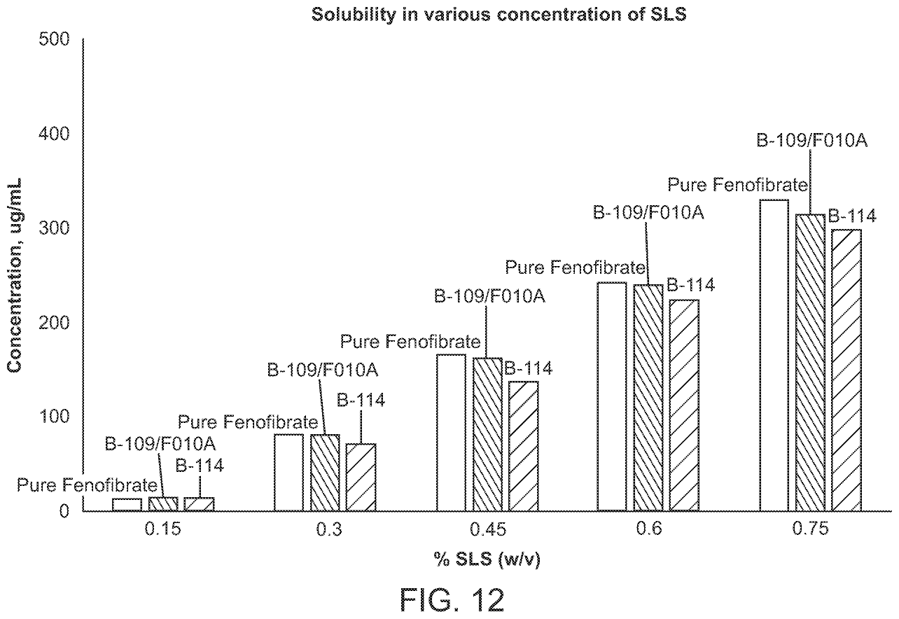

FIG. 12 shows the solubility in solutions containing different amounts of SLS at room temperature. As shown in FIG. 12, at various concentrations of SLS, the solubility is largely the same for (1) uncoated particles, (2) zinc oxide coated particles and (3) zinc oxide plus silicon oxide coated particles.

### In Vivo Drug Release Analysis

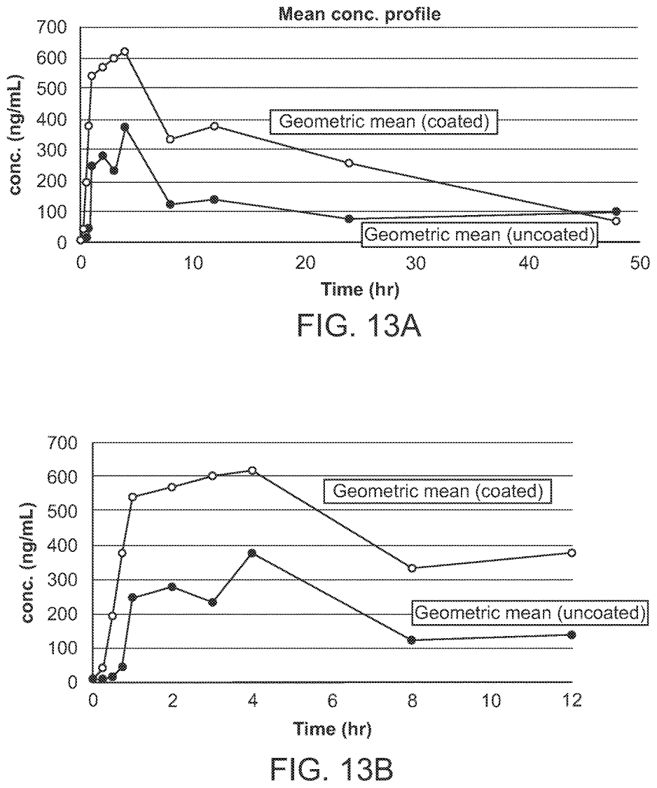

FIGS. 13A-13B show the results of the pharmacokinetic studies of zinc oxide plus silicon oxide coated particles in Beagle dogs (n=6). The detailed results are also shown in the table below. As shown in the table below, based on AUC, the zinc oxide plus silicon oxide coated particles showed a significant increase in bioavailability (~2 times) over the uncoated particles. The Cmax values indicated a significant increase (~3 times) over the uncoated particles. The Tmax values indicate quick availability of the API for absorption (i.e. resulting from improvement in dissolution rate).

TABLE 5
in vivo pharmacokinetic studies of zinc oxide plus
silicon oxide coated particles in Beagle dogs

| | | | | | Intra- |
|---|---|---|---|---|---|
| | Coated | Uncoated | Ratio | 90% CI for | subject |
| | Geometric Mean | | of Geo | ratio of | CV |
| | Arithmetic Mean (CV, %) | | Mean | Geo Mean | (%) |
| AUC0-t | 17495 | 7798, | 2.24 | 1.72 to 2.93 | 21.93 |
| | 19075 | 8688, | | | |
| | (48.58) | (49.12) | | | |
| Cmax | 1201, | 430 | 2.79 | 2.02 to 3.86 | 26.74 |
| | 1548 | 497 | | | |
| | (87.7) | (57.2) | | | |
| Tmax | 2 | 2.5 | | | |
| | (0.75-24) | (1-4) | | | |

FIG. 16 shows the results of the pharmacokinetic studies of zinc oxide plus silicon oxide coated particles in Wister rats (n=6). The coated and uncoated fenofibrate particles were administered weekly (one administration each week) by oral gavage over 28 days. The detailed results are also shown in the table below. As shown in the table below, based on AUC, the zinc oxide plus silicon oxide coated particles showed a significant increase in bioavailability (~3 times) over the uncoated particles. The Cmax values indicated a significant increase (~3 times) over the uncoated particles. The Tmax values indicate quick availability of the API for absorption (i.e. resulting from improvement in dissolution rate).

TABLE 6
in vivo pharmacokinetic studies of zinc oxide
plus silicon oxide coated particles in rats

| Pharmacokinetic | Coated API- | Uncoated API- | Improvement |
|---|---|---|---|
| parameters | High Dose (G3) | high dose (G5) | (multiples) |
| Cₘₐₓ(ng/ml) | 37418 | 11955 | 3.38 |
| Tₘₐₓ | 4 | 4 | Same |
| AUC | 459376.5 | 149164.6 | 3.08 |

### Stability Analysis

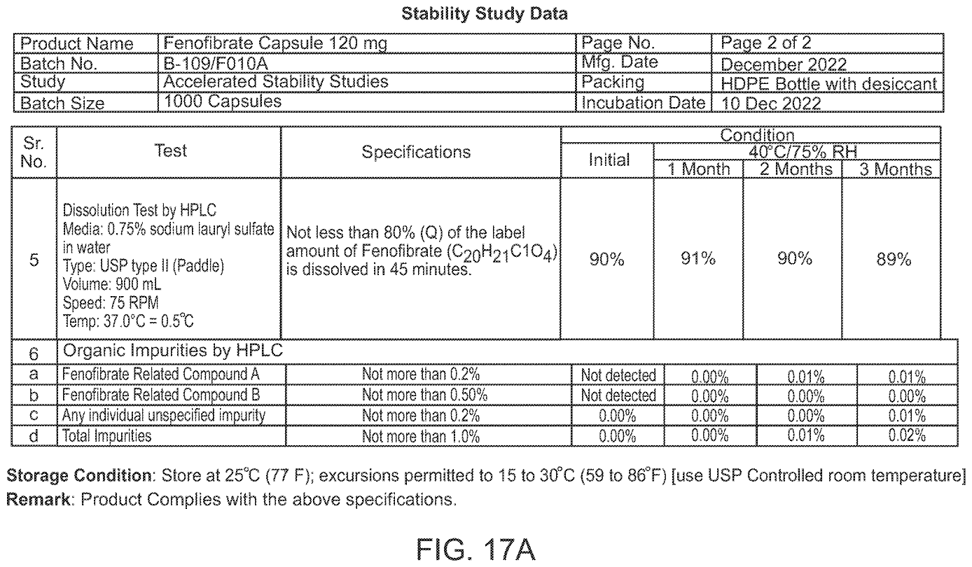

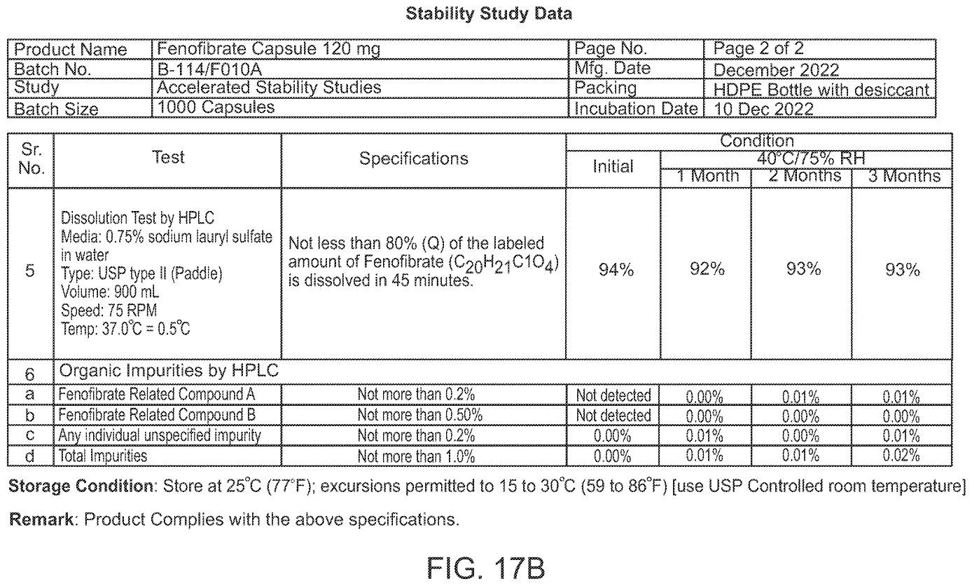

FIG. 17A-17B show the results from stability studies. FIG. 17A shows the results from uncoated fenofibrate particles. FIG. 17B shows the results from coated fenofibrate particles. The coated and uncoated fenofibrate particles were formulated into capsules, and stored in HDPE bottle with desiccant at 40° C. and 75% relative humidity for 1 month, 2 months, or 3 months. The samples were analyzed by a dissolution test following the parameters in FIGS. 17A-17B (Media: 0.75% SLS in water; Type: USP type II (Paddle); Volume: 900 ml; Speed: 75 RPM; Temperature: 37+/−0.5° C.). As shown in FIG. 17A, more than 80% of the labeled Fenofibrate was dissolved in 45 minutes. Overall, comparing to uncoated particles, more fenofibrate was released by the coated particles after 1 month, 2 months, and 3 months storage.

### Toxicity Analysis

The coated and uncoated fenofibrate particles were dispersed in 1% Carboxymethylcellulose (CMC) solution and administered to mice (n=6, 3 male and 3 female) by oral gavage repeatedly over 28 days.

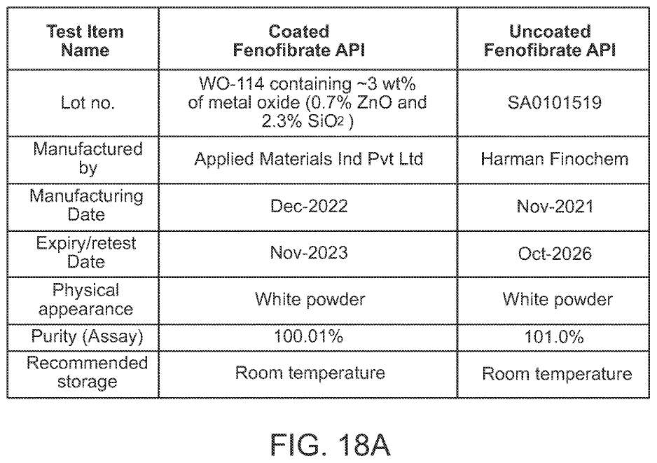

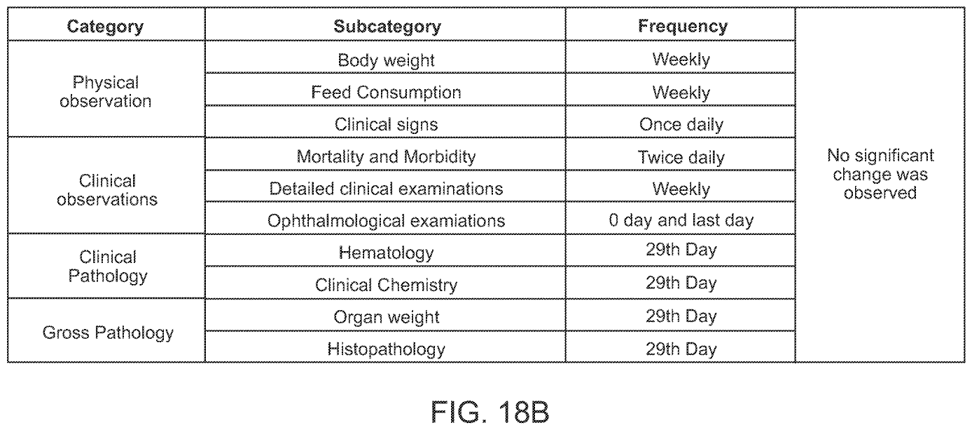

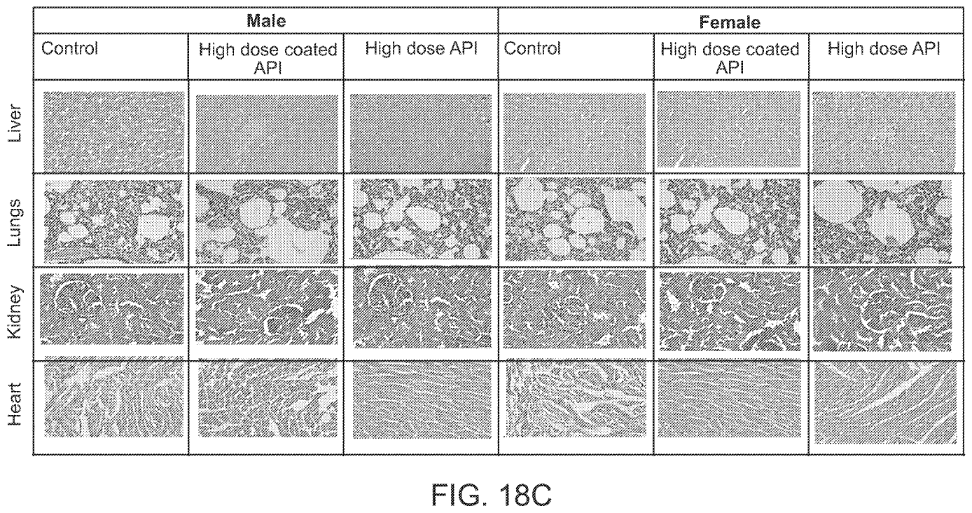

FIGS. 18A-18C show the results from toxicity studies. FIG. 18A shows the properties (e.g., physical appearance) of the coated and uncoated fenofibrate particles. FIG. 18B shows the study protocol and the results. FIG. 18C shows the histopathology images of major organs (including the liver, the lung, the kidney, and the heart) after administration of the coated and uncoated fenofibrate particles on the 29^{th} day. As shown in FIG. 18B, no significant change was observed in terms of physical observation (body weight, feed consumption and clinical signs), clinical observations (mortality and morbidity, detailed clinical examinations, ophthalmological examinations), clinical pathology (hematology, clinical chemistry), and gross pathology (organ weight, histopathology). Thus, the data indicate that the coated and uncoated fenofibrate particles were not toxic.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition, comprising:
(a) a coated particle comprising an active pharmaceutical ingredient (API)-containing core and one or more discontinuous metal oxide or metalloid oxide coating layers, wherein the API-containing core has a median particle size, on a volume average basis, between 0.1 μm and 1000 μm, and
(b) a pharmaceutically acceptable excipient.

2. The composition of claim 1, wherein the coated particle further comprises a continuous metal oxide or metalloid oxide coating layer.

3. The composition of claim 1, wherein the one or more discontinuous coating layers, taken together, do not provide a continuous pin-hole free coating of the API-containing core.

4. The composition of claim 1, wherein the API-containing core has a median particle size, on a volume average basis, between 0.1 μm and 20 μm.

5. The composition of claim 1, wherein the API is a BCS class II molecule or a BCS class IV molecule.

6.-8. (canceled)

9. The composition of claim 1, wherein the metal oxide or metalloid oxide coating layers consist of zinc oxide and/or silicone oxide.

10. The composition of claim 1, wherein the one or more metal oxide or metalloid oxide coating layers comprise or consist of zinc oxide.

11. The composition of claim 1, wherein the one or more metal oxide or metalloid oxide coating layers comprise or consist of silicon oxide.

12. The composition of claim 1, wherein each of the one or more metal oxide or metalloid oxide coating layers consist of zinc oxide or silicon oxide.

13. The composition of claim 1, wherein the one or more metal oxide or metalloid oxide coating layers comprise a discontinuous inner coating layer that is selected from zinc oxide and titanium oxide and an outer coating layer that is silicon oxide.

14. The composition of claim 1, wherein the one or more metal oxide or metalloid oxide coating layers, taken together constitute 2%-7% wt/wt of the coated particles.

15.-21. (canceled)

22. The composition of claim 1, wherein the uncoated particles have a water contact angle that is between 90° and 145°.

23. The composition of claim 1, wherein the uncoated particles have a water contact angle that is greater than 145°.

24. The composition of claim 1, wherein the coated particles have a water contact angle that is less than 90°.

25. A method of preparing coated particles comprising active pharmaceutical ingredient (API)-containing core, a discontinuous zinc oxide coating layer and a discontinuous silicon oxide coating layer, the method comprising the sequential steps of:
(a) loading particles comprising an API into the chamber of a reactor, wherein the particles have a median particle size, on a volume average basis between 0.1 μm and 1000 μm;
(b1) applying vaporous or gaseous diethyl zinc to the particles in the reactor by pulsing the vaporous or gaseous diethyl zinc into the reactor;
(b2) performing one or more pump-purge cycles of the reactor using an inert gas;
(b3) applying vaporous or gaseous oxidant to the particles in the reactor by pulsing the oxidant into the reactor;
(b4) performing one or more pump-purge cycles of the reactor using an inert gas;
(c) repeating steps (b1)-(b4) at least once to create a discontinuous zinc oxide coating layer on the particles;
(d1) applying vaporous or gaseous silicon tetrachloride to the particles in the reactor by pulsing the vaporous or gaseous silicon precursor into the reactor;
(d2) performing one or more pump-purge cycles of the reactor using an inert gas;
(d3) applying vaporous or gaseous water to the particles in the reactor by pulsing vaporous or gaseous water into the reactor;
(d4) performing one or more pump-purge cycles of the reactor using an inert gas; and
(e) repeating steps (d1)-(d4) at least once to create a discontinuous silicon oxide coating layer on the discontinuous zinc oxide coating layer.

26. (canceled)

27. (canceled)

28. The method of claim 25, wherein the uncoated particles have a water contact angle that is between 90° and 145°.

29. The method of claim 25, wherein the uncoated particles have a water contact angle that is greater than 145°.

30. The method of claim 25, wherein the coated particles have a water contact angle that is less than 90°.

31.-33. (canceled)

34. The method of claim 25, wherein the discontinuous coating layers, taken together do not provide a continuous pin-hole free coating of the API-containing core.

35. The method of claim 25, wherein the one or more metal oxide or metalloid oxide coating layers, taken together constitute 2%-7% wt/wt of the coated particles.

36.-39. (canceled)
